(19) United States
(12) Patent Application Publication
(10) Pub. No.: US 2024/0240226 A1
(43) Pub. Date: Jul. 18, 2024

(54) FLUOROGENIC ASSAY FOR RAPID SCREENING OF BACTERIAL BETA-LACTAMASE ACTIVITY

(71) Applicant: The Board of Trustees of the Leland Stanford Junior University, Stanford, CA (US)

(72) Inventors: Jianghong RAO, Palo Alto, CA (US); Jinghang XIE, Fremont, CA (US); Ran MU, Stanford, CA (US)

(21) Appl. No.: 18/550,786

(22) PCT Filed: Apr. 8, 2022
(86) PCT No.: PCT/US2022/024071

§ 371 Date: Sep. 15, 2023

Related U.S. Application Data
(60) Provisional application No. 63/172,321, filed on Apr. 8, 2021.

(51) Int. Cl. C12Q 1/18; C07D 265/38; C07D 477/14; C07D 501/36; C12Q 1/34; G01N 21/64

(52) U.S. Cl. CPC C12Q 1/18; C07D 265/38; C07D 477/14; C07D 501/36; C12Q 1/34; G01N 21/6428; G01N 2021/6439; G01N 2333/986

(57) ABSTRACT

A dual-caged fluorogenic resorufin probe (CDA) has been developed that is stable under physiological condition with low background but becomes highly fluorescent upon β-lactamase/esterase activation and further oxidation. The probes of the disclosure are advantageous for initial screening of broad-spectrum β-lactam antibiotics resistance and carbapenem resistant pathogens at diagnosis. After a two-step filtration, the assay of the disclosure can report 10³ c.f.u./mL cephalosporin- and carbapenem-resistant bacteria in urine within 2 hours at room temperature.

### CROSS-REFERENCE TO RELATED APPLICATION

This application claims the benefit of U.S. Provisional Application No. 63/172,321 filed on Apr. 8, 2021, titled “FLUOROGENIC ASSAY FOR RAPID SCREENING OF BACTERIAL BETA-LACTAMASE ACTIVITY” the entire disclosure of which is incorporated herein by reference.

### STATEMENT REGARDING FEDERALLY SPONSORED RESEARCH OR DEVELOPMENT

This invention was made with Government support under contract Al125286 awarded by the National Institutes of Health. The Government has certain rights in the invention.

### TECHNICAL FIELD

The present disclosure is generally related to compositions comprising a beta-lactamase- or carbapenemase-sensitive fluorescent probe and methods of synthesis thereof. The present disclosure is also generally related to a method of detecting a beta-lactamase-or carbapenemase-resistant bacterial strain.

### BACKGROUND

Superbugs could kill as many as 10 million people each year globally by 2050, nearly 2 million more than cancer. The lack of pre-prescription antimicrobial susceptibility testing (AST) that can inform the burden of drug resistant pathogenic bacteria, such as rapid and accurate diagnostics to identify common resistance mechanisms, has increased unnecessary antimicrobial usage, delayed pathogen-specific infection management, and allowed continued spreading of drug resistance.

Discovered nearly a century ago, β-lactam antibiotics (e.g., penicillins, cephalosporins and carbapenems) remain the most successful class of antimicrobials, constituting 60% of worldwide antibiotic usage, and are among the most effective agents for treatment of infections. One major acquirable mechanism for β-lactam resistance is the production of β-lactamases which break down β-lactams to metabolites incapable of binding to PBPs (Penicillin-binding proteins). From 2010 to 2014, the rates of extended-spectrum β-lactamase (ESBL)-positive Escherichia coli (E. coli) isolated in urinary specimens from across the United States increased from 7.9 to 18.3%; the activity of all tested cephalosporin antibiotics decreased significantly by about 9%. Isolates carrying carbapenemases, the enzyme that could hydrolyze nearly all β-lactam antibiotics including the last-resort carbapenem antibiotics, are also on a rising trend.

Genotypic nucleic acid testing (NAT) like FISH (fluorescence in situ hybridization) and PCR have been used for expeditious detection of β-lactamase genes. But NAT has major limitations. First, it is nearly impossible to develop a NAT that can cover all β-lactamase genes and mutations, considering that more than one thousand distinct β-lactamases have been identified in natural clinical isolates. Second, the detection of nucleic acid does not predict the persistence of viable pathogens, as the inactivation of pathogens by antibiotics often triggers a slow decay of cellular components including cytoplasmic membrane. Third, the mere presence of genetic materials do not necessarily imply their expression or function, and vice versa, poses significant challenges in typing strains or isolates with real antimicrobial resistance and toxin production. Emerging diagnostic platforms are employing microfluidics, biosensors, and lab-on-a-chip technology for genotypic and phenotypic detection of β-lactamases. Rapid phenotypic ASTs hybridized with microfluidic chips and biosensors measuring optical, chemical, electrical, mechanical, or other signals during bacteria growth in the presence of β-lactam antibiotics have been demonstrated even with very low numbers of bacteria and sample volumes and in parallel testing of different pathogens with multiple antibiotics. While holding great potentials, their robustness, cost-effectiveness, and adaptability into large scale clinical setting remains to be further illustrated. Development of a rapid screening assay for β-lactamase activity is critical for combating lactam antibiotic resistance and enhancing antibiotics stewardship.

### SUMMARY

Embodiments of the present disclosure provide for a dual-caged fluorogenic resorufin probe (CDA) has been developed that is stable under physiological condition with low background but becomes highly fluorescent upon β-lactamase/esterase activation and further oxidation. The cephalosporin moiety renders a wide detection spectrum of β-lactamase expressing bacteria including cephalosporinases and carbapenemases, thus CDA is suitable for initial screening of broad-spectrum β-lactam antibiotics resistance and carbapenem resistant pathogens at diagnosis. After a simple two-step filtration, the CDA/H₂O₂ assay of the disclosure can report 10³ c.f.u./mL cephalosporin- and carbapenem-resistant bacteria in urine within 2 hours at room temperature. This cascade activation of dual-caged CDA and the filtration-based assay is advantageous for the rapid diagnosis of lactam-resistant bacterial pathogens and timely selection of appropriate treatment and prevent further spread of antibiotic resistance.

One aspect of the disclosure, Accordingly, one aspect of the disclosure encompasses embodiments of a dual-caged cleavable probe comprising a β-lactamase- or carbapenemase-cleavable moiety conjugated to a self-immolative linker, wherein the self-immolative linker is conjugated to a fluorescently detectable label.

In some embodiments of this aspect of the disclosure, the probe can have the formula:
R₁-β-lactam-self-immolative linker-fluorescently detectable label, wherein R₁ can be an H, a halogen, a substituent selected from NO₂, CN, carboxyl, OH, an amine, a substituted or unsubstituted alkyl, a substituted or unsubstituted aryl, a substituted or unsubstituted cycloalkyl, a substituted or unsubstituted cycloalkenyl, a substituted or unsubstituted aryl, a substituted or unsubstituted biaryl, a substituted or unsubstituted fused aryl, a substituted or unsubstituted alkenyl, or a substituted or unsubstituted alkynyl; the β-lactam can be a cephalosporin or a carbapenem, wherein the cephalosporin comprises the structure (a) or (b) and the carbapenem can have the structure (c)

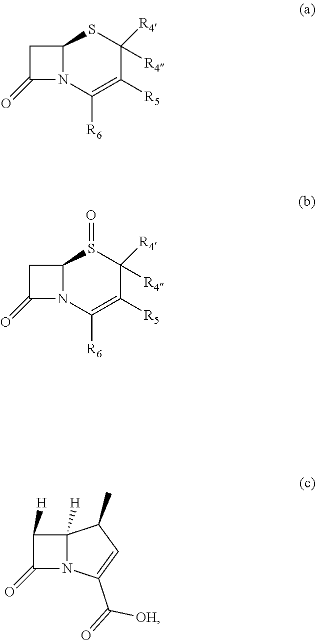

wherein R_{4′}, R_{4″}, R₅, and R₆ can be each independently a H, a halogen, a substituent selected from NO₂, CN, carboxyl, OH, an amine, a substituted or unsubstituted alkyl, a substituted or unsubstituted aryl, a substituted or unsubstituted cycloalkyl, a substituted or unsubstituted cycloalkenyl, a substituted or unsubstituted aryl, a substituted or unsubstituted biaryl, a substituted or unsubstituted fused aryl, a substituted or unsubstituted alkenyl, or a substituted or unsubstituted alkynyl; the self-immolative linker can have the structure:

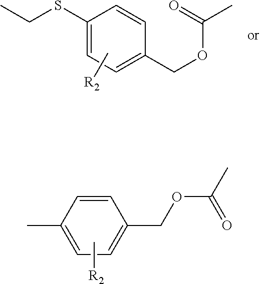

wherein R₂, can be a H, a halogen, a substituent selected from NO₂, CN, carboxyl, OH, an amine, a substituted or unsubstituted alkyl, a substituted or unsubstituted aryl, a substituted or unsubstituted cycloalkyl, a substituted or unsubstituted cycloalkenyl, a substituted or unsubstituted aryl, a substituted or unsubstituted biaryl, a substituted or unsubstituted fused aryl, a substituted or unsubstituted alkenyl, or a substituted or unsubstituted alkynyl. In some embodiments of this aspect of the disclosure, the fluorescently detectable label can have the structure:

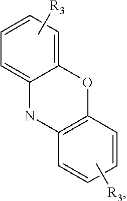

wherein R₃ can be a hydrogen, a halogen, a substituent selected from NO₂, CN, carboxyl, OH, an amine, a substituted or unsubstituted alkyl, a substituted or unsubstituted aryl, a substituted or unsubstituted cycloalkyl, a substituted or unsubstituted cycloalkenyl, a substituted or unsubstituted aryl, a substituted or unsubstituted biaryl, a substituted or unsubstituted fused aryl, a substituted or unsubstituted alkenyl, or a substituted or unsubstituted alkynyl.

In some embodiments of this aspect of the disclosure, the carbapenem can be selected from the group consisting of biapenem, ertapenem, doripenem, imipenem, and panipenem:

In some embodiments of this aspect of the disclosure, the probe can be a dual-caged N-cephalosporin-caged 3,7-diesterphenoxazine beta-lactamase-cleavable probe or a dual-caged carbapenem-caged 3,7-diesterphenoxazine carbapenemase-cleavable probe. In some embodiments of this aspect of the disclosure, the dual-caged N-cephalosporin-caged 3,7-diesterphenoxazine beta-lactamase-cleavable probe has the structure of formula I:

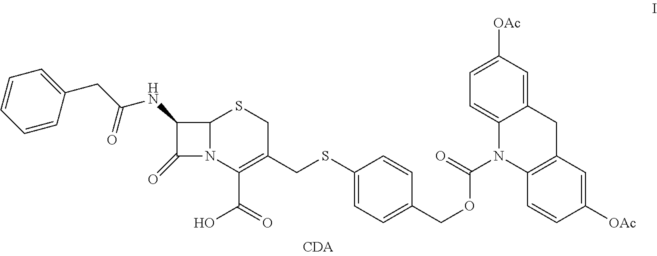 [SMILES: CC(=O)Oc1ccc2c(c1)Cc1cc(OC(C)=O)ccc1N2C(=O)OCc1ccc(SCC2=C(C(=O)O)N3C(=O)[C@@H](NC(=O)Cc4ccccc4)C3SC2)cc1]

In some embodiments of this aspect of the disclosure, the dual-caged carbapenem-caged 3,7-diesterphenoxazine carbapenemase-cleavable probe can have the structure of formula II:

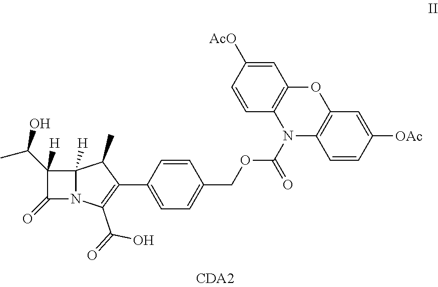 [SMILES: CC(=O)Oc1ccc2c(c1)Oc1cc(OC(C)=O)ccc1N2C(=O)OCc1ccc(C2=C(C(=O)O)N3C(=O)[C@H]([C@@H](C)O)[C@H]3[C@H]2C)cc1]

In some embodiments of this aspect of the disclosure, the detectable label is 10H-phenoxazine-3,7-diyl diacetate (2-DARR).

Another aspect of the disclosure encompasses embodiments of a method of detecting the presence of a bacterial strain that has resistance to at least one of a beta-lactam antibiotic or a carbapenem antibiotic, wherein said method comprises contacting a population of bacteria suspected of being resistant to at least one of a beta-lactam antibiotic or a carbapenem antibiotic with a dual-caged N-cephalosporin-caged 3,7-diesterphenoxazine beta-lactamase-cleavable probe or a dual-caged carbapenem-caged 3,7-diesterphenoxazine carbapenem-cleavable probe in the presence of an oxidizing agent, and measuring a fluorescent signal, wherein a detectable fluorescent signal indicates that the bacterial strain has at least one of a beta-lactamase or a carbapenemase.

In some embodiments of this aspect of the disclosure, the dual-caged N-cephalosporin-caged 3,7-diesterphenoxazine beta-lactamase-cleavable probe or a dual-caged carbapenem-caged 3,7-diesterphenoxazine carbapenem-cleavable probe can comprise β-lactamase- or carbapenemase-cleavable moiety conjugated to a self-immolative linker, wherein the self-immolative linker is conjugated to a fluorescently-detectable label moiety.

In some embodiments of this aspect of the disclosure, the fluorescently detectable label moiety is 10H-phenoxazine-3,7-diyl diacetate (2-DARR).

In some embodiments of this aspect of the disclosure, the dual-caged N-cephalosporin-caged 3,7-diesterphenoxazine beta-lactamase-cleavable probe has the structure of formula I:

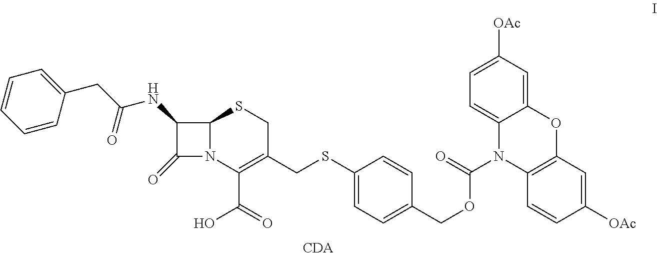 [SMILES: CC(=O)Oc1ccc2c(c1)Oc1cc(OC(C)=O)ccc1N2C(=O)OCc1ccc(SCC2=C(C(=O)O)N3C(=O)[C@@H](NC(=O)Cc4ccccc4)[C@H]3SC2)cc1]

and the dual-caged N-cephalosporin-caged 3,7-diesterphenoxazine beta-lactamase-cleavable probe has the structure of formula II:

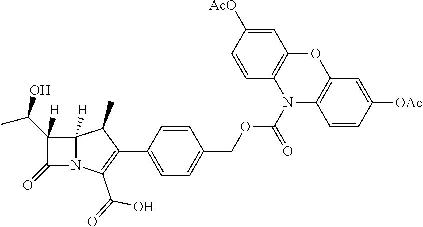 [SMILES: CC(=O)Oc1ccc2c(c1)Oc1cc(OC(C)=O)ccc1N2C(=O)OCc1ccc(C2=C(C(=O)O)N3C(=O)[C@H]([C@@H](C)O)[C@H]3[C@H]2C)cc1]

In some embodiments of this aspect of the disclosure, the oxidizing agent is hydrogen peroxide.

In some embodiments of this aspect of the disclosure, the method can further comprise culturing a biological sample from a human or animal to generate a population of bacteria from the sample and concentrating the population of cultured bacteria.

Yet another aspect of the disclosure encompasses embodiments of a method of synthesis of a dual-caged N-cephalosporin-caged 3,7-diesterphenoxazine beta-lactamase-cleavable probe having the structure of formula I:

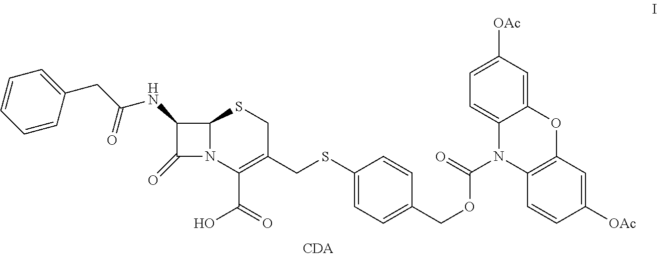 [SMILES: CC(=O)Oc1ccc2c(c1)Oc1cc(OC(C)=O)ccc1N2C(=O)OCc1ccc(SCC2=C(C(=O)O)N3C(=O)[C@@H](NC(=O)Cc4ccccc4)[C@H]3SC2)cc1]

wherein the method is according to Scheme 1 (FIG. 28) of the disclosure.

Still another aspect of the disclosure encompasses embodiments of a method of synthesis of a dual-caged N-cephalosporin-caged 3,7-diesterphenoxazine beta-lactamase-cleavable probe has the structure of formula II:

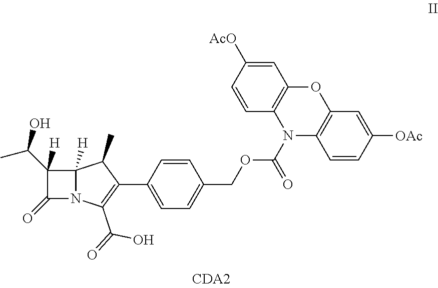 [SMILES: CC(=O)Oc1ccc2c(c1)Oc1cc(OC(C)=O)ccc1N2C(=O)OCc1ccc(C2=C(C(=O)O)N3C(=O)[C@H]([C@@H](C)O)[C@H]3[C@H]2C)cc1]

wherein the method is according to Scheme 2 (FIG. 29) of the disclosure.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 illustrates the design of CDA for targeting cephalosporinases and carbapenemases. a) Structure of CDA and its hydrolysis by β-lactamase (Bla) and esterase, followed by oxidation to render fluorescent resorufin. b) Fluorescent emission spectra (565-750 nm) of CDA, and CDA treated with esterase (1 μg/mL), TEM-1 Bla (100 nM), both enzymes with and without 1 mM H₂O₂ at room temperature for 8 hours (excitation, 525 nm). The activation of CDA results in a 1200-fold increase at emission 585 nm over CDA in PBS. Spectra were collected on a SpectraMax iD3 multimode microplate reader. A.U. indicates arbitrary units.

FIG. 2 illustrates the characterization of CDA with recombinant TEM-1 Bla, esterase, HRP, and H₂O₂. a) Stability test of CDA in different concentrations of H₂O₂ in PBS. The signal of PBS was subtracted and the absolute values were plotted. b) Fluorescence enhancement of CDA treated with esterase (1 g/mL), TEM-1 Bla (100 nM) and H₂O₂ (100 μM or 1 mM). c) Activation of CDA by esterase (1 μg/mL) and TEM-1 Bla (100 nM) with or without oxygen in the reaction. Glass flasks with working solution were continuously vacuumed to maintain a deoxidized environment. Samples were transferred and scanned immediate at each time point in a black 96 well plate. Error bars represent ±SD, n=3. d) Fluorescence enhancement of CDA treated with esterase (1 μg/mL), TEM-1 Bla (100 nM), HRP (1 unit/ml), and H₂O₂ (1 μM). e) Fluorescence enhancement of different concentrations of CDA treated with esterase (1 μg/mL), TEM-1 Bla (100 nM) and H₂O₂ (1 mM). f) Fluorescence enhancement of CDA treated with indicated amounts of β-lactamases, esterase (1 μg/mL) and H₂O₂ (1 mM) after incubation at room temperature for 2 h. The dash line represents the 3 times of the standard deviation of the mean value of negative control (PBS). All the studies were duplicated at room temperature in PBS (pH=7.05) as the buffer unless other indicated. The working concentration of CDA was 10 μM except e). Data were collected on a Tecan Safire microplate reader with excitation at 570 nm and emission at 600 nm. A.U. indicates arbitrary unit.

FIG. 3 illustrates the characterization of CDA with HRP, H₂O₂, E. coli expressing TEM-1 Bla (E. coli/TEM-1) and IMP-1 carbapenemase (E. coli/IMP-1), and K. pneumoniae expressing KPC carbapenemase (K. pneumoniae/KPC). a) Fluorescence enhancement of CDA incubated with different concentrations of E. coli expressing TEM-1 Bla. b) Fluorescence enhancement of CDA; HRP (1 unit/mL), H₂O₂ (1 μM), and either viable E. coli expressing TEM-1 Bla or bacterial lysate extracted from equal c.f.u. per milliliter. c) Fluorescence enhancement of CDA incubated with different concentrations of E. coli expressing TEM-1 Bla (H₂O₂, 1 mM). d) Fluorescent intensity of E. coli and E. coli/TEM-1 preincubated with CDA for 20 hours upon addition of H₂O₂ (500 μM) for 5 min. Error bars represent ±SD, n=3. * p<0.0332; **p<0.0021, ****p<0.0001, ns: not significant. e) Fluorescence enhancement of CDA incubated with different concentrations of E. coli expressing IMP-1 carbapenemase in the presence of H₂O₂ (1 mM). f) Fluorescence enhancement of CDA incubated with different concentrations of K. pneumoniae expressing KPC carbapenemase (H₂O₂, 1 mM). All the studies were duplicated at room temperature with PBS (pH=7.05) as the buffer unless other indicated. The working concentration of CDA was 10 μM. The signal of CDA in PBS (a), H₂O₂ 1 μM (b), or H₂O₂ 1 mM (c, e, f) was subtracted and the absolute values were plotted. Data were collected on a Tecan Safire microplate reader with excitation at 570 nm and emission at 600 nm. A.U. indicates arbitrary unit.

FIG. 4 illustrates the detection of lactam-resistant bacteria in urine samples. a) Illustration of the workflow in bacteria isolation and concentration from a urine sample for detection. The sample is first filtrated by a 5-μm filter to trap cells and large debris followed by a 0.22-μm filter to trap bacteria. The 0.22-μm filter is washed once before PBS/H₂O₂ (1 mM) is directed through the 0.22-μm filter in the opposite direction with a new syringe to recover the concentrated bacteria for detection in a plate reader. b) Longitudinal monitoring of fluorescence enhancement with CDA and concentrated E. coli, K. pneumoniae, K. pneumoniae/KPC, E. coli/TEM-1, E. coli/IMP-1, E. coli/KPC-3, E. cloacae/IMP, S. marcescens/SME, and E. coli/OXA-48 in the presence of H₂O₂ (1 mM). Star* indicate clinical isolates. The study was duplicated at room temperature with PBS as the buffer. The signal of CDA alone in PBS containing H₂O₂ (1 mM) was subtracted and the absolute values were plotted. The working concentration of CDA was 10 μM. Data were collected on a Tecan Safire microplate reader with excitation at 570 nm and emission at 600 nm. A.U. indicates arbitrary units.

FIG. 5 illustrates DARR, CDA, AR (1 mM in DMSO) and PBS in test tubes under white light.

FIG. 6 illustrates the fluorescence spectra of CDA and Amplex red. a) Fluorescent emission spectra of PBS, CDA, and AR (10 μM probes in PBS; excitation, 525 nm). b) Fluorescent emission spectra of CDA treated with esterase (1 μg/mL), TEM-1 Bla (100 nM) and H₂O₂ (1 mM) at room temperature for 8 hours. c) Fluorescent emission spectra of AR and AR treated with HRP and H₂O₂ at room temperature for 5 min (10 μM AR in PBS, HRP 1 unit/mL, H₂O₂ 1 uM) (excitation, 525 nm). A.U. indicates arbitrary units.

FIG. 7 illustrates an SDS-PAGE analysis of purified TEM-1 Bla. Lad: SeeBlue Plus 2 pre-stained protein standard; P: pellet of lysate; S: supernatant of lysate; F.T .: flow through fraction of supernatant from TALON resin column; Wash by HEPES buffer; Lanes 1-3: wash by 0, 10, or 20 mM imidazole; Lanes 4-14: elution from the TALON resin column with 40, 60, 80, 100, 120, 140, 160, 180, 200, 250 or 500 mM imidazole. The gel was stained by SimpleBlue staining reagent.

FIG. 8 illustrates the quantification of purified TEM-1 Bla. a) SDS-PAGE analysis of purified TEM-1 Bla and BSA as standard. Lad: SeeBlue Plus 2 pre-stained protein standard. The gel was stained by SimpleBlue staining reagent. Upper panel: scan under bright field; lower panel: scan at 800 nm by a LI-COR odyssey scanner and near-infrared intensity defined by regions of interest (ROIs). b) Standard curve generated by plotting near-infrared intensity and the amount of BSA loaded to normalize the concentration of TEM-1 Bla.

FIG. 9 illustrates the LC-MS analysis of CDA and CDA in 1 mM H₂O₂ at room temperature for 2 hours. a) HPLC traces of CDA with or without H₂O₂; b) Mass spectra of CDA with or without H₂O₂ in positive electrospray ionization modes. Peak * indicates CDA (calculated M.W. 795.83).

FIG. 10 illustrates the stability of DARR in H₂O₂. a) Structure of DARR and its oxidation by H₂O₂ into fluorescent resorufin. b) Stability test of DARR (10 μM) in different concentrations of H₂O₂ at room temperature. Data were collected on a Tecan Safire microplate reader with excitation at 570 nm and emission at 600 nm. A.U. indicates arbitrary units.

FIG. 11 illustrates that esterase sensitizes DARR in response to H₂O₂. a) Structure of DARR and its hydrolysis by esterase and oxidation into fluorescent resorufin by H₂O₂. b) Fluorescence enhancement of DARR treated with esterase (1 μg/mL) and different concentrations of H₂O₂ at room temperature. Data were collected on a Tecan Safire microplate reader with excitation at 570 nm and emission at 600 nm. A.U. indicates arbitrary units.

FIG. 12 illustrates the fluorescence change of CDA (10 μM) in the presence of esterase (1 μg/mL), TEM-1 Bla (100 nM), H₂O₂ (1 μM) and HRP (0.1 or 1 unit/mL) at room temperature in PBS (pH=7.05). Data were collected on a Tecan Safire microplate reader with excitation at 570 nm and emission at 600 nm. A.U. indicates arbitrary unit.

FIG. 13 illustrates a response of CDA (10 μM) to different concentration of H₂O₂ in the presence of recombinant TEM-1 Bla (100 nM) and esterase (1 μg/mL) at room temperature in PBS (pH=7.05). Data were collected on a Tecan Safire microplate reader with excitation at 570 nm and emission at 600 nm. A.U. indicates arbitrary unit.

FIG. 14 illustrates an SDS-PAGE analysis of purified AmpC. Lad: SeeBlue Plus 2 pre-stained protein standard; P: pellet of lysate; S: supernatant of lysate; F.T .: flow through fraction of supernatant from TALON resin column; Wash by HEPES buffer-1 and HEPES buffer containing 10 mM imidazole-2; Lanes 3-13: elution from the TALON resin column with 20, 40, 60, 80, 100, 120, 140, 160, 180, 200 or 250 mM imidazole. The gel was stained by SimpleBlue staining reagent and scanned with a LI-COR Odyssey.

FIG. 15 illustrates an SDS-PAGE analysis of purified OXA-48. Lad: SeeBlue Plus 2 pre-stained protein standard; P: pellet of lysate; S: supernatant of lysate; F.T .: flow through fraction of supernatant from TALON resin column; Wash by HEPES buffer-1 and HEPES buffer containing 10 mM imidazole-2; Lanes 3-13: elution from the TALON resin column with 20, 40, 60, 80, 100, 120, 140, 160, 180, 200 or 250 mM imidazole. The gel was stained by SimpleBlue staining reagent and scanned with a LI-COR Odyssey.

FIG. 16 illustrates an SDS-PAGE analysis of purified KPC-3. Lad: SeeBlue Plus 2 pre-stained protein standard; P: pellet of lysate; S: supernatant of lysate; F.T .: flow through fraction of supernatant from TALON resin column; Wash by HEPES buffer-1 and HEPES buffer containing 10 mM imidazole-2; Lanes 3-13: elution from the TALON resin column with 20, 40, 60, 80, 100, 120, 140, 160, 180, 200 or 250 mM imidazole. The gel was stained by SimpleBlue staining reagent and scanned with a LI-COR Odyssey.

FIG. 17 illustrates the quantification of purified AmpC, OXA-48 and KPC-3. a) SDS-PAGE analysis of purified AmpC, OXA-48, KPC-3 and BSA as standard. Lad: SeeBlue Plus 2 pre-stained protein standard. The gel was stained by SimpleBlue staining reagent. The gel was scanned at 800 nm by a LI-COR odyssey scanner and near-infrared intensity was defined by regions of interest (ROIs). b) Standard curve generated by plotting near-infrared intensity and the amount of BSA loaded to normalize the concentration of β-lactamases.

FIG. 18 illustrates an SDS-PAGE analysis of purified IMP-1. Lad: SeeBlue Plus 2 pre-stained protein standard; P: pellet of lysate; S: supernatant of lysate; F.T .: flow through fraction of supernatant from TALON resin column; Wash by HEPES buffer-1 and HEPES buffer containing 10 mM imidazole-2; Lanes 3-14: elution from the TALON resin column with 20, 40, 60, 80, 100, 120, 140, 160, 180, 200, 250 and 500 mM imidazole. The gel was stained by SimpleBlue staining reagent and scanned with a LI-COR Odyssey.

FIG. 19 illustrates a quantification of purified IMP-1. a) SDS-PAGE analysis of purified IMP-1 and BSA as standard. Lad: SeeBlue Plus 2 pre-stained protein standard. The gel was stained by SimpleBlue staining reagent. Upper panel: scan under bright field; lower panel: scan at 800 nm by a LI-COR odyssey scanner and near-infrared intensity defined by regions of interest (ROIs). b) Standard curve generated by plotting near-infrared intensity and the amount of BSA loaded to normalize the concentration of IMP-1.

FIG. 20 illustrates a calibration of the optical density (OD) and E.coli colony-forming unit (c.f.u.). Left: the correlation of c.f.u./mL calculated from OD600 absorbance to colony count/mL on agar plates. Right: a representative agar plate with around 100 colonies from 100 pL of 103 c.f.u./mL E. coli suspension determined by measuring its absorbance at OD600.

FIG. 21 illustrates the fluorescence enhancement of CDA with different concentrations of E. coli with or without H₂O₂. The study was duplicated at room temperature with PBS (pH=7.05) as the buffer. The working concentration of CDA was 10 μM. The signal of CDA in PBS (a) or H₂O₂ (b) was subtracted and the absolute values were plotted. Data were collected on a Tecan Safire microplate reader with excitation at 570 nm and emission at 600 nm. A.U. indicates arbitrary unit.

FIG. 22 illustrates the fluorescence change of CDA with different concentrations of E. coli expressing TEM-1 Bla in the presence of H₂O₂ (100 μM, 500 μM, 2 mM). The signal of CDA in corresponding concentration of H₂O₂ were subtracted and the absolute values were plotted. All the studies were performed at room temperature in PBS (pH=7.05). The working concentration of CDA was 10 μM. Data were collected on a Tecan Safire microplate reader with excitation at 570 nm and emission at 600 nm. A.U. indicates arbitrary unit.

FIG. 23 illustrates the fluorescence enhancement of CDA incubated with different concentrations of E. coli expressing KPC-3 carbapenemase in PBS at room temperature; CDA, 10 μM, and H₂O₂, 1 mM. The signal of CDA in H₂O₂ was subtracted and the absolute values were plotted. Data were collected on a Tecan Safire microplate reader with excitation at 570 nm and emission at 600 nm. A.U. indicates arbitrary units.

FIG. 24 illustrates the fluorescence enhancement of CDA incubated with different concentrations of noninduced E. coli/TEM-1 in PBS at room temperature; CDA, 10 μM and H₂O₂, 1 mM. The signal of CDA in H₂O₂ was subtracted and the absolute values were plotted. Data were collected on a Tecan Safire microplate reader with excitation at 570 nm and emission at 600 nm. A.U. indicates arbitrary units.

FIG. 25 illustrates the fluorescence enhancement of CDA (10 μM) incubated with different concentrations of imipenem sensitive K. pneumoniae in PBS at room temperature (H₂O₂, 1 mM). The signal of CDA in H₂O₂ was subtracted and the absolute values were plotted. Data were collected on a Tecan Safire microplate reader with excitation at 570 nm and emission at 600 nm. A.U. indicates arbitrary units.

FIG. 26 illustrates a kit used for urinary bacterial detection. The kit contains 1) a 0.22-μm polypropylene filter, 2) a 5-μm polypropylene filter, 3) a 20-mL sterile syringe, 4) a 1-mL sterile syringe, and 5) a 50-mL sterile conical tube.

FIG. 27 illustrates the comparison of nylon and polypropylene filters in concentrating bacteria from urine samples. Designated volume from 100 mL synthetic urine spiked with E. coli/TEM-1 Bla at 103 c.f.u./mL (top), filtrate after 5-μm filters (middle), and recovered bacteria in PBS (bottom) were spread on agar-based LB medium to monitor the efficiency of filtration and concentration.

FIG. 28 illustrates Scheme 1 for the synthesis of CDA. a) i. Zn/AcOH, RT; ii. Acetone, DMAP, Ac₂O, RT. b) i. Triphosgene/TEA; ii. DCE. c) mCPBA, DCM, 0° C. to rt. d) K₂CO₃, CH₃CN. e) K₂CO₃/DMAP, DCE, Ar, RT. f) Nal/TFAA, acetone, 0° C., Ar. g) TFA/TIPS, DCM, RT. RT=room temperature, DMAP=4-dimethylaminopyridine, TEA=triethanolamine, mCPBA=meta-chloroperoxybenzoic acid, TFAA=trifluoroacetic anhydride, DCE=1,2-dichloroethane, TFA=trifluoroacetic acid, DCM=dichloromethane, TIPS=triisopropylsilane.

FIG. 29 illustrates Scheme 2 for the synthesis of CDA2. a) TBS-CI, imidazole, DMF, RT, 5 h, 90%. b) Rh₂(OAc)₄, ZnCl₂, DCM, reflux, 2 h. c) HTMP, DIPEA, Tf₂O, −50° C., 2.5 h. d) TMSCI, Nal, MeCN, 0° C. to RT, 30 min. e) K₂CO₃, DMF, RT. f) Triflate, Pd(OAc)₂, K₂CO₃, DMF, 37° C. g) NH₄HF₂, NMP/DMF. h) Zn dust, PB (pH=6.0).

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, microbiology, molecular biology, pharmacology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Room temperature is defined as 20-23° C. Standard pressure is defined as 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

### Abbreviations

ESBL, extended broad-spectrum beta-lactamases; TFAA, Trifluoroacetic anhydride; MLB, metallo-β-lactamase; CRE, carbapenem-resistant enterobacteriaceae; PBS, Phosphate-buffered saline; CPBA, chloroperoxybenzoic acid; TFAA, trifluoroacetic anhydride; TFA, trifluoroacetic acid; TIPS, SDS, sodium dodecyl sulfate

### Definitions

In describing and claiming the disclosed subject matter, the following terminology will be used in accordance with the definitions set forth below.

The term “caging group” as used herein refers to a moiety that can be employed to reversibly block, inhibit, or interfere with the activity (e.g., the biological activity) of a molecule (e.g., a polypeptide, a nucleic acid, a small molecule, a drug, and the like). Typically, one or more caging groups are associated (covalently or noncovalently) with the molecule but do not necessarily surround the molecule in a physical cage. Caging groups can be, for example, relatively small moieties such as carboxyl nitrobenzyl, 2-nitrobenzyl, nitroindoline, hydroxyphenacyl, DMNPE, quinilones including bromoquinilones and derivatives thereof, or the like, or they can be, e.g., large bulky moieties such as a protein or a bead. Caging groups can be removed from a molecule, or their interference with the molecule's activity can be otherwise reversed or reduced, by exposure to an appropriate type of uncaging energy and/or exposure to an uncaging chemical, enzyme, or the like. The caging groups of the present disclosure may be released from the blocked or “caged” nucleoside triphophoester (uncoupled) by photolysis following two-photon excitation.

The term “detectable moiety” as used herein refers to a label molecule (isotopic or non-isotopic) which is incorporated indirectly or directly into a liposomal nanoparticle according to the disclosure, wherein the label molecule facilitates the detection of the nanoparticle in which it is incorporated. Thus, “detectable moiety” is used synonymously with “label molecule”. Label molecules, known to those skilled in the art as being useful for detection, include chemiluminescent or fluorescent molecules. Various fluorescent molecules are known in the art which are suitable for use to label a nucleic acid for the method of the present invention. The protocol for such incorporation may vary depending upon the fluorescent molecule used. Such protocols are known in the art for the respective fluorescent molecule.

The term “fluorescence” as used herein refers to a luminescence that is mostly found as an optical phenomenon in cold bodies, in which the molecular absorption of a photon triggers the emission of a photon with a longer (less energetic) wavelength. The energy difference between the absorbed and emitted photons ends up as molecular rotations, vibrations or heat. Sometimes the absorbed photon is in the ultraviolet range, and the emitted light is in the visible range, but this depends on the absorbance curve and Stokes shift of the particular fluorophore.

The term “fluorophore” as used herein refers to any reporter group whose presence can be detected by its light emitting properties.

The term “operably linked” as used herein refers to an arrangement of elements wherein the components so described are configured so as to perform their usual function.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “self-immolative probe” as used herein refers to a signaling molecule covalently bound to a moiety (a “self-immolative arm” or “self-immolative linker”) such that the self-immolative arm inhibits the signaling molecule from signaling. The self immolative arm is covalently bound to an reporter moiety such as a fluorophore of the disclosure such that the removal of a moiety by the action of an enzyme, for example, causes a destabilization of the self-immolative arm such that the self immolative arm becomes removed from the signaling molecule, allowing the signaling molecule to signal. The “self-immolative” moieties of the disclosure include such as a substrate group of an enzyme. For example, but not intended to be limiting a self-immolative linker may have attached thereon a phosphate group. A cellular phosphatase may, on contact with a probe of the disclosure, cleave the phosphate group from the linker, thereby allowing the liner to reconfigure to allow electron transfer to the fluorophore to emit a detectable signal.

The term “beta-lactamase” as used herein refers to enzymes that hydrolyze the beta-lactam ring of the beta-lactam antibiotics. According to Ambler (Philos. Trans. R. Soc. London Ser B, (1980) 289: 321-331), beta-lactamases are classified in 4 groups: A: penicillinases, including extended broad-spectrum beta-lactamases (ESBLs); B: metallo-enzymes; cephalosporinases; D: oxacillinases.

Hydrolysis of the amide bond of the beta-lactam ring makes the antimicrobial agents biologically inactive. Class A beta-lactamases (Ambler classification) refer to serine beta-lactamases, in which hydrolysis of beta-lactam is mediated by serine in the active site, usually at amino acid position 70 in the alpha helix₂.

The term “carbapenemases” as used herein are a diverse group of beta-lactamases that are active not only against the oxyimino-cephalosporins and cephamycins but also against the carbapenems. Carbapenemase may be a metallo-beta-lactamase or a serine-beta-lactamase. Broad spectrum carbapenemases can be selected from, for example, an IMP-type carbapenemases (metallo-beta-lactamases), VIMs (Verona integron-encoded metallo-beta-lactamases), OM (oxacillinase) group of beta-lactamases, KPCs (Klebsiella pneumonia carbapenemases), CMY (Class C), SME, IMI, NMC, GES (Guiana extended spectrum), CcrA, SFC-1, SHV-38, and NDM (New Delhi metallo-beta-lactamases, e.g. NDM-1) beta-lactamases.

The term “alkyl”, either alone or within other terms such as “thioalkyl” and “arylalkyl”, means a monovalent, saturated hydrocarbon radical which may be a straight chain (i.e. linear) or a branched chain. An alkyl radical for use in the present disclosure generally comprises from about 1 to 20 carbon atoms, particularly from about 1 to 10, 1 to 8 or 1 to 7, more particularly about 1 to 6 carbon atoms, or 3 to 6. Illustrative alkyl radicals include methyl, ethyl, n-propyl, n-butyl, n-pentyl, n-hexyl, isopropyl, isobutyl, isopentyl, amyl, sec-butyl, tert-butyl, tert-pentyl, n-heptyl, n-actyl, n-nonyl, n-decyl, undecyl, n-dodecyl, n-tetradecyl, pentadecyl, n-hexadecyl, heptadecyl, n-octadecyl, nonadecyl, eicosyl, dosyl, n-tetracosyl, and the like, along with branched variations thereof. In certain aspects of the disclosure an alkyl radical is a C₁-C₆ lower alkyl comprising or selected from the group consisting of methyl, ethyl, n-propyl, n-butyl, n-pentyl, n-hexyl, isopropyl, isobutyl, isopentyl, amyl, tributyl, sec-butyl, tert-butyl, tert-pentyl, and n-hexyl. An alkyl radical may be optionally substituted with substituents as defined herein at positions that do not significantly interfere with the preparation of compounds of the disclosure and do not significantly reduce the efficacy of the compounds. In certain aspects of the disclosure, an alkyl radical is substituted with one to five substituents including halo, lower alkoxy, lower aliphatic, a substituted lower aliphatic, hydroxy, cyano, nitro, thio, amino, keto, aldehyde, ester, amide, substituted amino, carboxyl, sulfonyl, sulfuryl, sulfenyl, sulfate, sulfoxide, substituted carboxyl, halogenated lower alkyl (e.g. CF₃), halogenated lower alkoxy, hydroxycarbonyl, lower alkoxycarbonyl, lower alkylcarbonyloxy, lower alkylcarbonylamino, cycloaliphatic, substituted cycloaliphatic, or aryl (e.g., phenylmethyl benzyl)), heteroaryl (e.g., pyridyl), and heterocyclic (e.g., piperidinyl, morpholinyl). Substituents on an alkyl group may themselves be substituted.

The terms “cyclic” and “cycloalkyl” as used herein refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur, or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

The term “aryloxyl” as used herein refers to an aryl-O—group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

The term “alkenyl” as used herein refers to an unsaturated, acyclic branched or straight-chain hydrocarbon radical comprising at least one double bond. An alkenyl radical may contain from about 2 to 24 or 2 to 10 carbon atoms, in particular from about 3 to 8 carbon atoms and more particularly about 3 to 6 or 2 to 6 carbon atoms. Suitable alkenyl radicals include without limitation ethenyl, propenyl (e.g., prop-1-en-1-yl, prop-1-en-2-yl, prop-2-en-1-yl (allyl), and prop-2-en-2-yl), buten-1-yl, but-1-en-2-yl, 2-methyl-prop-1-en-1-yl, but-2-en-1-yl, but-2-en-2-yl, buta-1,3-dien-1-yl, beta-1,3-dien-2-3/1, hexen-1-yl, 3-hydroxyhexen-yl, hepten-1-yl, and octen-1-yl, and the like. An alkenyl radical may be optionally substituted similar to alkyl.

The term “substituted alkenyl” as used herein includes an alkenyl group substituted by, for example, one to three substituents, preferably one to two substituents, such as alkyl, alkoxy, haloalkoxy, alkylalkoxy, haloalkoxyalkyl, alkanoyl, alkanoyloxy, cycloalkyl, cycloalkoxy, acyl, acylamino, acyloxy, amino, alkylamino, alkanoylamino, aminoacyl, aminoacyloxy, cyano, halogen, hydroxyl, carboxyl, carboxylalkyl, carbamyl, keto, thioketo, thiol, alkylthio, sulfonyl, sulfonamido, thioalkoxy, aryl, nitro, and the like.

The term “alkynyl” as used herein refers to an unsaturated, branched or straight-chain hydrocarbon radical comprising one or more triple bonds. An alkynyl radical may contain about 1 to 20, 1 to 15, or 2 to 10 carbon atoms, particularly about 3 to 8 carbon atoms and more particularly about 3 to 6 carbon atoms. Suitable alkynyl radicals include without limitation ethynyl, such as prop-1-yn-1-yl and prop-2-yn-1-yl, butyryls such as but-1-yn-1-yl, but-1-yn-3-yl, and but-3-yn-1-yl, pentynyls such as perityn-1-yl, pentyn-2-yl, 4-methoxypentyn-2-yl, and 3-methylbutyn-1-yl, hexynyls such as hexyn-1-yl, hexyn-2-yl, hexyn-3-yl, and 3,3-dimethylbutyn-1-yl radicals and the like. In aspects of the disclosure, alkenyl groups include ethenyl (—CH═CH₂), n-propenyl (—CH₂CH═CH₂), iso-propenyl (—C(CH₃)═CH₂), and the like. An alkynyl may be optionally substituted similar to alkyl. The term “cycloalkynyl” refers to cyclic alkynyl groups.

The term “substituted alkynyl” as used herein includes an alkynyl group substituted by, for example, a substituent, such as, alkyl, alkoxy, alkanoyl, alkanoyloxy, cycloalkyl, cycloalkoxy, acyl, acylamino, acyloxy, amino, alkylamino, alkanoylamino, aminoacyl, aminoacyloxy, cyano, halogen, hydroxyl, carboxyl, carboxylalkyl, carbamyl, keto, thioketo, thiol, alkylthio, sulfonyl, sulfonamido, thioalitoxy, aryl, nitro, and the like. The term “alkylene” as used herein refers to a linear or branched radical having from about 1 to 10, 1 to 8, 1 to 6, or 2 to 6 carbon atoms and having attachment points for two or more covalent bonds. Examples of such radicals are methylene, ethylene, propylene, butylene, pentylene, hexylene, ethylidene, methylethylene, and isopropylidene. When an alkenylene radical is present as a substituent on another radical it is typically considered to be a single substituent rather than a radical formed by two substituents.

The term “alkenylene” as used herein refers to a linear or branched radical having from about 2 to 10, 2 to 8 or 2 to 6 carbon atoms, at least one double bond, and having attachment points for two or more covalent bonds. Examples of alkenylene radicals include 1,1-vinylidene (—CH₂═C—), 1,2-vinylidene (—CH═CH—), and 1,4-butadienyl (—CH═CH—CH═CH—). The term “halo” as used herein refers to a halogen such as fluorine, chlorine, bromine or iodine atoms.

The term “hydroxyl” or “hydroxy” as used herein refers to mm —OH group.

The term “cyano” as used herein refers to a carbon radical having three of four covalent bonds shared by a nitrogen atom, in particular —CN. A cyano group may be substituted with substituents described herein.

The term “alkoxy” refers to a linear or branched oxy-containing radical having an alkyl portion of one to about ten carbon atoms, such as a methoxy radical, which may be substituted. In aspects of the disclosure an alkoxy radical may comprise about 1-10, 1-8, 1-6 or 1-3 carbon atoms. In embodiments of the disclosure, an alkoxy radical comprises about 1-6 carbon atoms and includes a C₁-C₆ alkyl-O-radical wherein C₁-C₆ alkyl has the meaning set out herein. Examples a alkoxy radicals include without limitation methoxy, ethoxy, propoxy, butoxy, isopropoxy and tert-butoxy alkyls. An “alkoxy” radical may, optionally be substituted with one or more substitutents disclosed herein including alkyl atoms to provide “alkylalkoxy” radicals; halo atoms, such as fluoro, chloro or bromo, to provide “haloalkoxy” radicals (e.g. fluoromethoxy, chloromethoxy, trifluoromethoxy, difluoromethoxy, trifluoroethoxy, fluoroethoxy, tetrafluoroethoxy, pentafluoroethoxy, and fluoropropox) and “haloalkoxyalkyl” radicals (e.g. fluoromethoxymethyl, chloromethoxyethyl, trifluorornethoxymethyl, difluoromethoxyethyl, and trifluorocthoxymethyl).

The term “alkenyloxy” as used herein refers to linear or branched oxy-containing radicals having an alkenyl portion of about 2 to 10 carbon atoms, such as an ethenyloxy or propenyloxy radical. An alkenyloxy radical may be a “lower alkenyloxy” radical having about 2 to 6 carbon atoms. Examples of alkenyloxy radicals include without limitation ethenyloxy, propenyloxy, butenyloxy, and isopropenyloxy alkyls. An “alkenyloxy” radical may be substituted with one or more substitutents disclosed herein including halo atoms, such as fluoro, chloro or bromo, to provide “haloalkenyloxy” radicals (e.g. trifluoroethenyloxy, fluoroethenyloxy, difluoroethenyloxy, and fluoropropenyloxy).

The term “carbocylic” as used herein includes radicals derived from a saturated or unsaturated, substituted or unsubstituted 5 to 14 member organic nucleus whose ring forming atoms (other than hydrogen) are solely carbon. Examples of carbocyclic radicals are cycloalkyl, cycloalkenyl, aryl, in particular phenyl, naphthyl, norbornanyl, bicycloheptadienyl, toluoyl, xylenyl, indenyl, stilbenzyl, terphenylyl, diphenylethylenyl, phenylcyclohexyl, acenapththylenyi, anthracenyl, biphenyl, bibenzylyl, and related bibenzylyl homologs, octahydronaphthyl, tetrahydronaphthyl, octahydroquinolinyl, dimethoxytetrahydronaphthyl and the like.

The term “cycloalkyl” as used herein refers to radicals having from about 3 to 15, 3 to 10, 3 to 8, or 3 to 6 carbon atoms and containing one, two, three, or four rings wherein such rings may be attached in a pendant manner or may be fused. In aspects of the disclosure, “cycloalkyl” refers to an optionally substituted, saturated hydrocarbon ring system containing 1 to 2 rings and 3 to 7 carbons per ring which may be further fused with an unsaturated C3-C7 carbocylic ring. Examples of cycloalkyl groups include single ring structures such as cyclopropyl, cyclobutyl, cyclopentyl, cyclohexyl, cycloheptyl, cyclooctyl, cyclononyl, cyclodecyl, cyclododecyl, and the like, or multiple ring structures such as adamantanyl, and the like. Tin certain aspects of the disclosure the cycloalkyl radicals are “lower cycloalkyl” radicals having from about 3 to 10, 3 to 8, 3 to 6, or 3 to 4 carbon atoms, in particular cyclopropyl, cyclobutyl, cyclopentyl, cyclohexyl and cycloheptyl. The term “cycloalkyl” also embraces radicals where cycloalkyl radicals are fused with aryl radicals or heterocyclyl radicals. A cycloalkyl radical may be optionally substituted with groups as disclosed herein.

The term “substituted cycloalkyl” as used herein includes cycloalkyl groups having from 1 to 5 (in particular 1 to 3) substituents including without limitation alkyl, alkenyl, alkoxy, cycloalkyl, substituted cycloalkyl, acyl, acylamino, acyloxy, amino, aminoacyl, aminoacyloxy, oxyacylamino, cyano, halogen, hydroxyl, carboxyl, carboxylalkyl, keto, thioketo, thiol, thioalkoxy, aryl, aryloxy, heteroaryl, heteroaryloxy, hydroxyamino, alkoxyamino, and nitro.

The term “cycloaliphatic” refers to a cycloalkane possessing less than 8 carbons or a fused ring system consisting of no more than three fused cycloaliphatic rings. Examples of such groups include, but are not limited to, decalin and the like.

The term “substituted cycloaliphatic” as used herein refers to a cycloalkane possessing less than 8 carbons or a fused ring system consisting of no more than three fused rings, and where at least one of the aliphatic hydrogen atoms has been replaced by a halogen, a nitro, a thio, an amino, a hydroxy, a ketone, an aldehyde, an ester, an amide, a lower aliphatic, a substituted lower aliphatic, or a ring (aryl, substituted aryl, cycloaliphatic, or substituted cycloaliphatic). Examples of such groups include, but are not limited to, 1-chlorodecalyl and the like.

The term “cycloalkenyl” as used herein refers to radicals comprising about 4 to 16, 2 to 15, 2 to 10, 2 to 8, 4 to 10, 3 to 8, 3 to 7, 3 to 6, or 4 to 6 carbon atoms, one or more carbon-carbon double bonds, and one, two, three, or four rings wherein such rings may be attached in a pendant manner or may be fused. In certain aspects of the disclosure the cycloalkenyl radicals are “lower cycloalkenyl” radicals having three to seven carbon atoms. Examples of cycloalkenyl radicals include without limitation cyclobutenyl, cyclopentenyl, cyclohexenyl and cycloheptenyl. A cycloalkenyl radical may be optionally substituted with groups as disclosed herein, in particular 1, 2, or 3 substituents which may be the same or different.

The term “aryl” as used herein refers to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

The term “aralkyloxyl” as used herein refers to an aralkyl-O—group wherein the aralkyl group is as previously described. An exemplary aralkyloxyl group is benzyloxyl. The term “substituted aryl” as used herein includes an aromatic ring, or fused aromatic ring system consisting of no more than three fused rings at least one of which is aromatic, and where at least one of the hydrogen atoms on a ring carbon has been replaced by a halogen, an amino, a hydroxy, a nitro, a thio, an alkyl, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, hydroxyphenyl, chlorphenyl and the like.

The term “aralkyl” refers to an aryl or a substituted aryl group bonded directly through an alkyl group, such as benzyl. Other particular examples of substituted aryl radicals include chlorobenzyl, and amino benzyl.

The term “aryloxy” as used herein refers to aryl radicals, as defined above, attached to an oxygen atom. Exemplary aryloxy groups include napthyloxy, quinolyloxy, isoquiriolizinyloxy, and the like.

The term “arylalkoxy” as used herein refers to an aryl group attached to an alkoxy group. Representative examples of arylalkoxy groups include, but are not limited to, 2-phenylethoxy, 3-naphth-2-ylpropoxy, and 5-phenylpentyloxy.

The term “heteroaryl” as used herein refers to fully unsaturated heteroatom-containing ring-shaped aromatic radicals having at least one heteroatom selected from carbon, nitrogen, sulfur and oxygen. A heteroaryl radical may contain one, two or three rings and the rings may be attached in a pendant manner or may be fused. In aspects of the disclosure the term refers to fully unsaturated hetoreatom-containing ring-shaped aromatic radicals having from 3 to 15, 3 to 10, 3 to 8, 5 to 15, 5 to 10, or 5 to 8 ring members selected from carbon, nitrogen, sulfur and oxygen, wherein at least one ring atom is a heteroatom. Examples of “heteroaryl” radicals, include without limitation, an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, tetrazolyl and the like; an unsaturated condensed heterocyclic group containing 1 to 5 nitrogen atoms, in particular, indolyl, isoindolyl, indolizinyl, benzimidazolyl, quinolyl, isoquinolyl, indazolyl, quinazolinyl, pteridinyl, quinolizidinyl, phthalazinyl, naphthyridinyl, quinoxalinyl, cinnolinyl, phenanthridinyl, acridinyl, phenanthrolinyl, phenazinyl, carbazolyl; purinyl, benzimidazolyl, quinolinyl, isoquinolinyl, beazotriazolyl, tetrazolopyridazinyl and the like; an unsaturated 3 to 6-membered heteromonocyclic group containing an oxygen atom, in particular, 2-furyl, pyranyl, and the like; an unsaturated 5 to 6-membered heteromonocyclic group containing a sulfur atom, in particular, thienyl, 2-thienyl, 3-thienyl, and the like; unsaturated 5 to 6-membered heteromonocyclic group containing 1 to 2 oxygen atoms and 1 to 3 nitrogen atoms, in particular, furazanyl, benzofurazanyl, oxazolyl, isoxazolyl, and oxadiazolyl; an unsaturated condensed heterocyclic group containing 1 to 2 oxygen atoms and 1 to 3 nitrogen atoms, in particular benzoxazolyl, benzoxadiazolyl and the like; an unsaturated 5 to 6-membered heteromonocyclic group containing 1 to 2 sulfur atoms and 1 to 3 nitrogen atoms, for example, thiazolyl, isothiazolyl, thiadiazolyl and the like; an unsaturated condensed heterocyclic group containing 1 to 2 sulfur atoms and 1 to 3 nitrogen atoms such as benzothiazolyl, benzothiadiazolyl and the like. The term also includes radicals where heterocyclic radicals are fused with aryl radicals, in particular bicyclic radicals such as benzofuranyl, benzothiophenyl, phthalazinyl, chromenyl, xanthenyl, and the like. A heteroaryl radical may be optionally substituted with groups as disclosed herein, for example with an alkyl, amino, halogen, etc., in particular a heteroarylamine. The term may refer to an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, pyridinyl, pyrimidinyl, pyrazinyl, pyridazinyl, triazolyl, tetrazolyl and the like. A heteroaryl radical may be optionally substituted with groups disclosed herein, for example with an alkyl, amino, halogen, etc., in particular a substituted heteroaryl radical is a heteroarylamine.

The term “heterocyclic” as used herein refers to a cycloalkane and/or an aryl ring system, possessing less than 8 carbons, or a fused ring system consisting of no more than three fused rings, where at least one of the ring carbon atoms is replaced by oxygen, nitrogen or sulfur. Examples of such groups include, but are not limited to, molpholino and the like.

The term “substituted heterocyclic” as used herein refers to a cycloalkane and/or an aryl ring system, possessing less than 8 carbons, or a fused ring system consisting of no more than three fused rings, where at least one of the ring carbon atoms is replaced by oxygen, nitrogen or sulfur, and where at least one of the aliphatic hydrogen atoms has been replaced by a halogen, hydroxy, a thio, nitro, an amino, a ketone, an aldehyde, an ester, an amid; a lower aliphatic, a substituted lower aliphatic, or a ring (aryl, substituted aryl, cycloaliphatic, or substituted cycloaliphatic). Examples of such groups include, but are not limited to 2-chloropyranyl.

The foregoing heteroaryl and heterocyclic groups may be C-attached or N-attached (where such is possible).

The term “sulfoxide” refers to the radical —S═O.

The term “amino” as used herein, alone or in combination, refers to a radical where a nitrogen atom (N) is bonded to three substituents being any combination of hydrogen, hydroxyl, alkyl, cycloalkyl, alkenyl, alkynyl, aryl, silyl, heterocyclic, or heteroaryl which may or may not be substituted. Term “thiol” as used herein means -SH. A thiol may be substituted with a substituent disclosed herein, in particular alkyl (thioalkyl), aryl (thioaiyl), alkoxy (thioalkoxy) or carboxyl.

The term “thioalkyl” as used herein, alone or in combination, refers to a chemical functional group where a sulfur atom (5) is bonded to an alkyl, which may be substituted. Examples of thioalkyl groups are thiomethyl, thioethyl, and thiopropyl. A thioalkyl may be substituted with a substituted or unsubstituted carboxyl, aryl, heterocylic, carbonyl, or heterocyclic.

The term “thioaryl” as used herein, alone or in combination, refers to a chemical functional group where a sulfur atom (S) is bonded to an aryl group with the general chemical formula —SR₂₃ where R₂₃ is aryl which may be substituted. Illustrative examples of thioaryl groups and substituted thioaryl groups are thiophenyl, chlorothiophenol, para-chlorothiophenol, thiobenzyl, 4-methoxy-thiophenyl, 4-nitro-thiophenyl, and para-nitrothiobenzyl.

The term “thioalkoxy” as used herein, alone or in combination, refers to a chemical functional group where a sulfur atom (S) is bonded to an alkoxy group with the general chemical formula —SR₂₄ where R₂₄ is an alkoxy group which may be substituted. A “thioalkoxy group” may have 1-6 carbon atoms i.e. a —S—(O)—C₁-C₆ alkyl group wherein C₁-C₆ alkyl have the meaning as defined above. Illustrative examples of a straight or branched thioalkoxy group or radical having from 1 to 6 carbon atoms, also known as a C₁-C₆ thioalkoxy, include thiomethoxy and thioethoxy.

A thiol may be substituted with a substituted or unsubstituted heteroaryl or heterocyclic, in particular a substituted or unsubstituted saturated 3 to 6-membered heteromonocylic group containing 1 to 4 nitrogen atoms [e.g. pyrrolidinyl, imidazolidinyl, piperidinyl, and piperazinyl] or a saturated 3 to 6-membered heteromonocyclic group containing 1 to 2 oxygen atoms and 1 to 3 nitrogen atoms [e.g. morpholinyl; sydrionyl], especially a substituted morpholinyl or piperidinyl.

The term “carbonyl” as used herein refers to a carbon radical having two of the four covalent bonds shared with an oxygen atom.

The term “carboxyl” as used herein, alone or in combination, refers to —C(O)OR₂₅— or —C(—O)OR₂₅ wherein R₂₅ is hydrogen, alkyl, alkenyl, alkynyl, cycloalkyl, cycloalkenyl, amino, thiol, aryl, heteroaryl, thioalkyl, thioaryl, thioalkoxy, a heteroaryl, or a heterocyclic, which may optionally be substituted. In aspects of the disclosure, the carboxyl groups are in an esterified form and may contain as an esterifying group lower alkyl groups. In particular aspects of the disclosure, —C(O)OR₂₅ provides an ester or an amino acid derivative. An esterified form is also particularly referred to herein as a “carboxylic ester”. In aspects of the disclosure a “carboxyl” may be substituted, in particular substituted with allyl which is optionally substituted with one or more of amino, amine, halo, alkylamino, aryl, carboxyl, or a heterocyclic. Examples of carboxyl groups are methoxycarbonyl, butoxycarbonyl, tert.alkoxycarbonyl such as tert.butoxycarbonyl, arylmethyoxycarbonyl having one or two aryl radicals including without limitation phenyl optionally substituted by for example lower alkyl, lower alkoxy, hydroxyl, halo, and/or nitro, such as benzyloxycarbonyl, methoxybenzyloxycarbonyl, diphenylmethoxycarbonyl, 2-bromoethoxycarbonyl, 2-iodoethoxycarbonyltert.butylcarborlyl, 4-nitrobenzyloxycarbonyl, diphenylmethoxy-carbonyl, benzhydroxycarbonyl, di-(4-methoxyphenyl-methoxycarbonyl, 2-bromoethoxycarbonyl, 2-iodoethoxycarbonyl, 2-trimethylsilylethoxycarbonyl, or 2-triphenylsilylethoxycarbonyl. Additional carboxyl groups in esterified form are silyloxycarbonyl groups including organic silyloxycarbonyl. The silicon substituent in such compounds may be substituted with lower alkyl (e.g. methyl), alkoxy (e.g. methoxy), and/or halo (e.g. chlorine). Examples of silicon substituents include trimethylsilyi and dimethyltert.butylsilyl. In aspects of the disclosure, the carboxyl group may be an alkoxy carbonyl, in particular methoxy carbonyl, ethoxy carbonyl, isopropoxy carbonyl, t-butoxycarbonyl, t-pentyloxycarbonyl, sir heptyloxy carbonyl, especially methoxy carbonyl or ethoxy carbonyl.

The term “carbamoyl” as used herein, alone or in combination, refers to amino, monoalkylamino, dialkylamino, monocycloalkylamino, alkyleycloalkylamino, and dicycloalkylaxaino radicals, attached to one of two unshared bonds in a carbonyl group.

The term “dialkylamino” as used herein refers to an —NRR′ group wherein each of R and R′ is independently an alkyl group and/or a substituted alkyl group as previously described. Exemplary alkylamino groups include ethylmethylamino, dimethylamino, and diethylamino.

The term “carboxamide” as used herein refers to the group —CONH—.

The term “nitro” as used herein means —NO₂—.

The terms “halo,” “halide,” or “halogen” as used herein refer to fluoro, chloro, bromo, and iodo groups.

The term “hydroxyl” as used herein refers to the —OH group.

The term “hydroxyalkyl” as used herein refers to an alkyl group substituted with an —OH group.

The term “mercapto” as used herein refers to the —SH group.

The term “oxo” as used herein refers to a compound described previously herein wherein a carbon atom is replaced by an oxygen atom.

The term “nitro” as used herein refers to the —NO₂ group.

The term “thio” as used herein refers to a compound described previously herein wherein a carbon or oxygen atom is replaced by a sulfur atom.

The term “sulfate” as used herein refers to the —SO₄ group.

The term “detectable moiety” as used herein refers to a label molecule (isotopic or non-isotopic) which is incorporated indirectly or directly into a liposomal nanoparticle according to the disclosure, wherein the label molecule facilitates the detection of the nanoparticle in which it is incorporated. Thus, “detectable moiety” is used synonymously with “label molecule”. Label molecules, known to those skilled in the art as being useful for detection, include chemiluminescent or fluorescent molecules. Various fluorescent molecules are known in the art which are suitable for use to label a nucleic acid for the method of the present invention. The protocol for such incorporation may vary depending upon the fluorescent molecule used. Such protocols are known in the art for the respective fluorescent molecule.

The term “dye” as used herein refers to any reporter group whose presence can be detected by its light absorbing or light emitting properties. For example, Cy5 is a reactive water-soluble fluorescent dye of the cyanine dye family. Cy5 is fluorescent in the red region (about 650 to about 670 nm). It may be synthesized with reactive groups on either one or both of the nitrogen side chains so that they can be chemically linked to either nucleic acids or protein molecules. Labeling is done for visualization and quantification purposes. Cy5 is excited maximally at about 649 nm and emits maximally at about 670 nm, in the far red part of the spectrum; quantum yield is 0.28. FW=792. Suitable fluorophores(chromes) for the probes of the disclosure may be selected from, but not intended to be limited to, fluorescein isothiocyanate (FITC, green), cyanine dyes Cy2, Cy3, Cy3.5, Cy5, Cy5.5 Cy7, Cy7.5 (ranging from green to near-infrared), Texas Red, and the like. Derivatives of these dyes for use in the embodiments of the disclosure may be, but are not limited to, Cy dyes (Amersham Bioscience), Alexa Fluors (Molecular Probes Inc.,), HILYTET Fluors (AnaSpec), DYLITE™ Fluors (Pierce, Inc), and resorufin.

The term “fluorescence” as used herein refers to a luminescence that is mostly found as an optical phenomenon in cold bodies, in which the molecular absorption of a photon triggers the emission of a photon with a longer (less energetic) wavelength. The energy difference between the absorbed and emitted photons ends up as molecular rotations, vibrations or heat. Sometimes the absorbed photon is in the ultraviolet range, and the emitted light is in the visible range, but this depends on the absorbance curve and Stokes shift of the particular fluorophore.

Spectroscopic, photochemical, biochemical, immunochemical, electrical, optical or chemical means can be used to detect such labels. The detection device and method may include, but is not limited to, optical imaging, electronic imaging, imaging with a CCD camera, integrated optical imaging, and mass spectrometry. Further, the amount of labeled or unlabeled probe bound to the target may be quantified. Such quantification may include statistical analysis.

Further definitions are provided in context below. Unless otherwise defined, all technical and scientific terms used herein have the same meaning as commonly understood by one of ordinary skill in the art of molecular biology. Although methods and materials similar or equivalent to those described herein can be used in the practice or testing of the present disclosure, suitable methods and materials are described herein.

### Discussion

The present disclosure encompasses embodiments of dual-caged novel N-cephalosporin caged probes useful in methods for detecting bacteria. The basic structure of the probes of the disclosure is:
R₁-β-lactam-self-immolative linker-fluorescently detectable label, wherein R₁ is H, a halogen, a substituent selected from NO₂, CN, carboxyl, OH, amine, a substituted or unsubstituted alkyl, a substituted or unsubstituted aryl, a substituted or unsubstituted cycloalkyl, a substituted or unsubstituted cycloalkenyl, a substituted or unsubstituted aryl, a substituted or unsubstituted biaryl, a substituted or unsubstituted fused aryl, a substituted or unsubstituted alkenyl, or a substituted or unsubstituted alkynyl;
the β-lactam is a cephalosporin or a carbapenem, wherein the cephalosporin comprises the structure (a) or (b) and the carbapenem has the structure (c) or variants thereof:

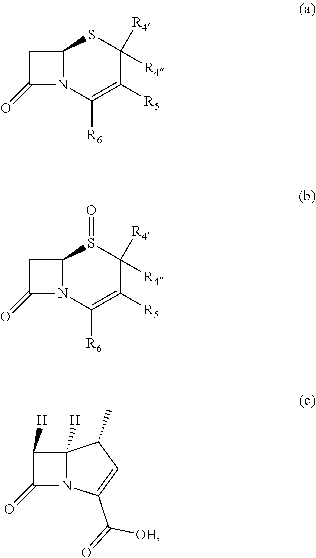

wherein R_{4′}, R_{4″}, R_{5,} and R₆ are each independently a H, a halogen, a substituent selected from NO₂, CN, carboxyl, OH, an amine, a substituted or unsubstituted alkyl, a substituted or unsubstituted aryl, a substituted or unsubstituted cycloalkyl, a substituted or unsubstituted cycloalkenyl, a substituted or unsubstituted aryl, a substituted or unsubstituted biaryl, a substituted or unsubstituted fused aryl, a substituted or unsubstituted alkenyl, or a substituted or unsubstituted alkynyl; the self-immolative linker has the structure:

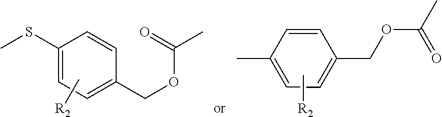

wherein R₂, is a H, a halogen, a substituent selected from NO₂, CN, carboxyl, OH, an amine, a substituted or unsubstituted alkyl, a substituted or unsubstituted aryl, a substituted or unsubstituted cycloalkyl, a substituted or unsubstituted cycloalkenyl, a substituted or unsubstituted aryl, a substituted or unsubstituted biaryl, a substituted or unsubstituted fused aryl, a substituted or unsubstituted alkenyl, or a substituted or unsubstituted alkynyl.

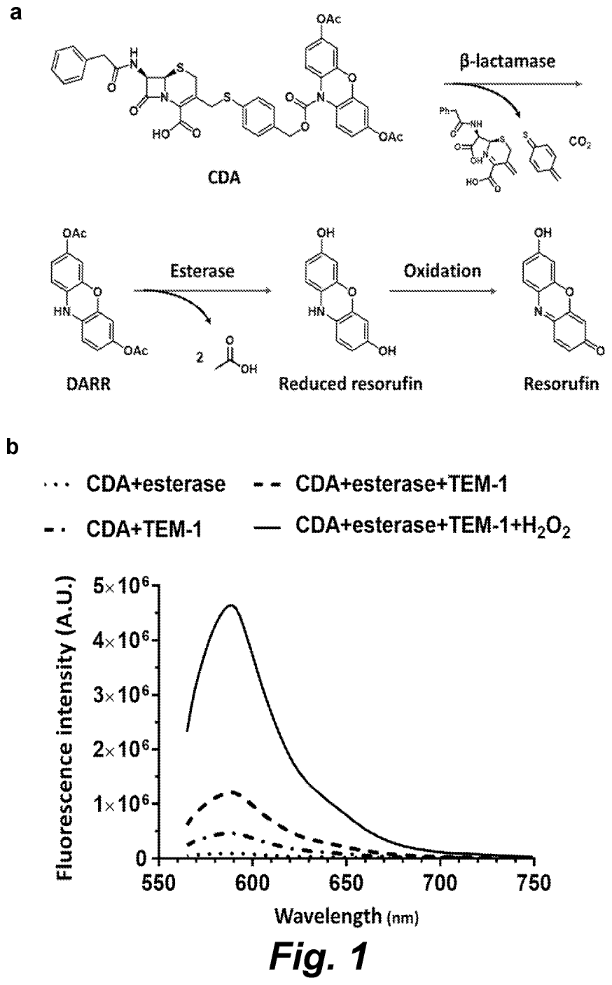

Exemplary embodiments of the probes include, but are not limiting, to a beta-lactamase positive 3,7-diesterphenoxazine probe (hereinafter termed CDA (Cephalosporin caged Diester Amplex red analogue)), the structure of which is shown in FIG. 1a and a fluorogenic assay for detecting β-lactamase expressing bacteria within hours of sample collection. As a highly fluorescent reporter (ϕ=0.75, pH=8), resorufin has excitation/emission at approximately 570/585 nm, and can be chemically caged for sensing enzyme activity (Dębski et al., Free Radical Biol. Med. (2016) 95: 323-332) for example, N-protected 3,7-dihydroxyphenoxazine (Han et al., Chemistry (2016) 11: 818-822; Hitomi et al., Anal. Chem. (2011) 83: 9213-9216; Hitomi et al., Chem. Commun. (Camb) (2013) 49: 9929-9931; Yadav et al., Angew Chem. Int. Ed. Engl. (2020) 59: 3307-3314) and O-alkyl resorufin derivatives (Gao et al., J. Am. Chem. Soc. (2003) 125: 11146-11147; Albers et al., Chem. Commun (Camb). (2007) DOI: 10.1039/b713190a, 4647-4649; Li et al., Analyt. Chem. (2013) 85: 3926-3932). As a precursor of a fluorescent probe, the amine-protected phenoxazine has overall low fluorescence background thus affording a high turn-on ratio. 10-Acetyl-3,7-dihydroxyphenoxazine (Amplex Red), for example, takes advantage of naturally low autofluorescence in biological samples and could determine hydrogen peroxide (H₂O₂) at nano- to picomolar levels.

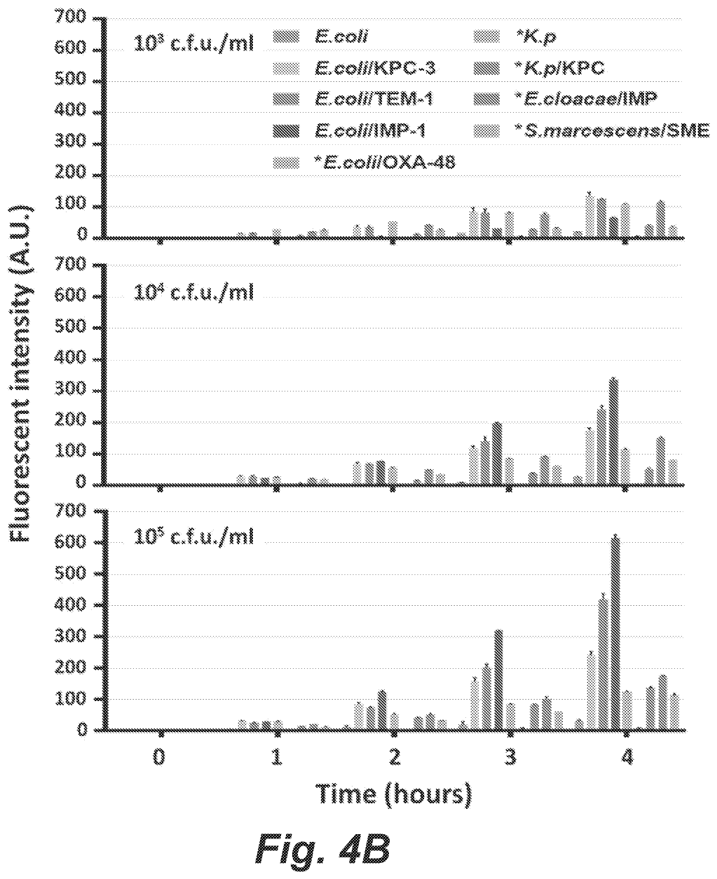

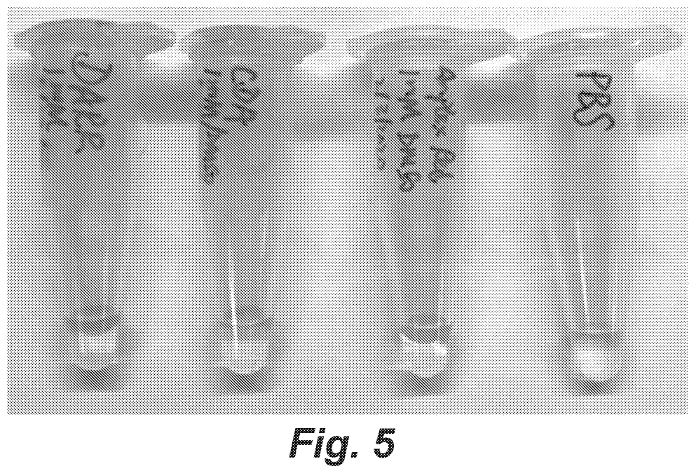

As described in the present disclosure, the amine was caged with an analogue of cefazolin, the first generation of cephalosporin antibiotics, which can be recognized by a broad spectrum of β-lactamases from cephalosporinases to carbapenemases. Esterification of the 3, 7 hydroxy groups allows better uptake by gram-negative bacteria and minimizes background, as shown in (FIGS. 5 and 6a). CDA can traverse the bacterial capsule either through passive diffusion, or more likely through active porin uptake of lactams. It then encounters β-lactamases within the periplasm of resistant bacteria where the cephalosporin moiety can be hydrolyzed to generate reduced diester resorufin (DARR). Continued hydrolysis of the diester bonds by universally present esterases in viable bacteria followed by oxidation then produces highly fluorescent resorufin (FIG. 1a). Without β-lactamases, CDA might still be processed by esterases but the product will remain non-fluorescent (FIG. 1b). Compared to singly caged fluorogenic probes, the cascade activation mechanism of the CDA of the disclosure can allow a lower background and greater detection sensitivity.

### Synthesis of CDA for Broad Spectrum β-Lactamases Detection

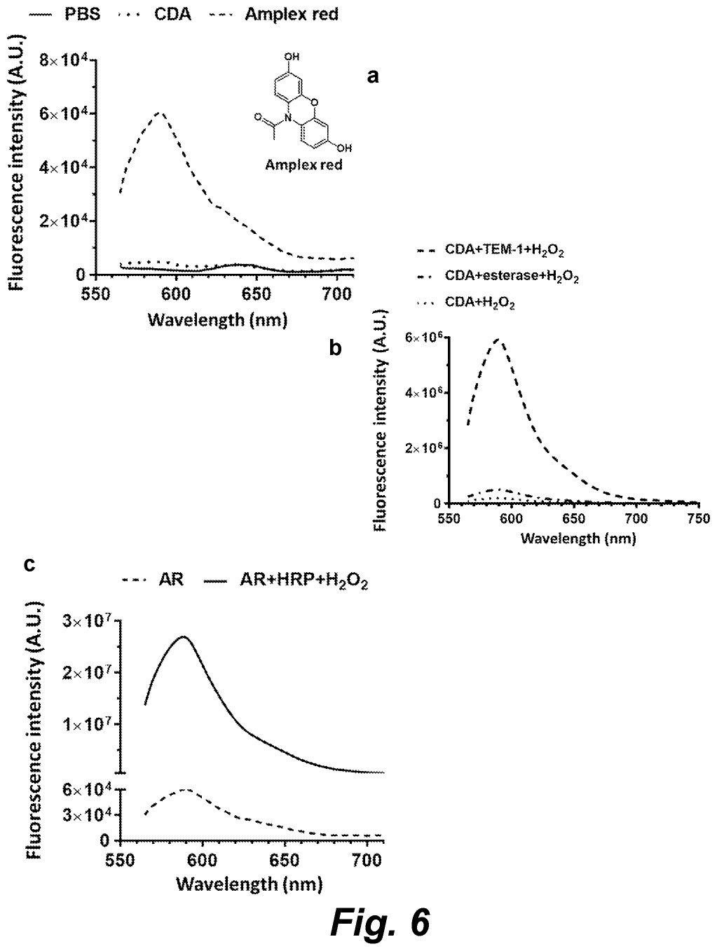

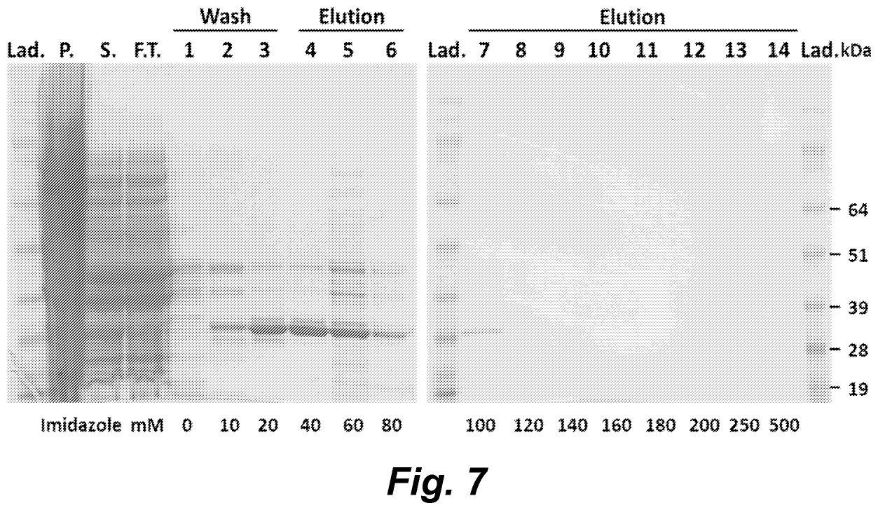

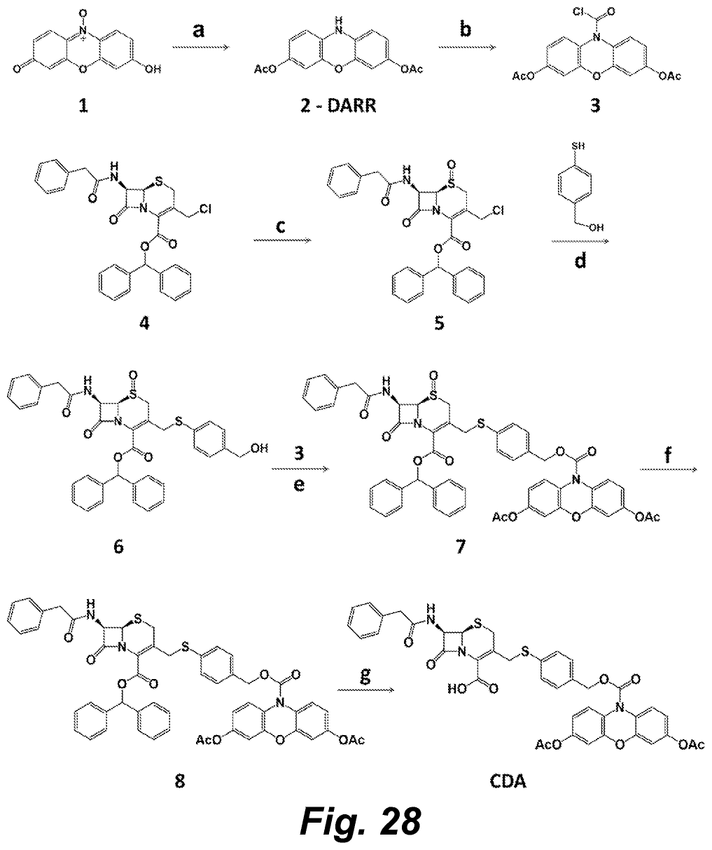

The synthesis of CDA is outlined in Scheme 1 (FIG. 28). DARR was prepared from resazurin sodium salt through reduction and acetylation (Han et al., Chemistry (2016) 11: 818-822) and conjugated to the oxidized cephalosporin chloride with a self-immolative linker 4-mercaptobenzyl alcohol to afford CDA after reduction and deprotection. When incubated with a recombinant class A cephalosporin-hydrolyzing β-lactamase TEM-1 (TEM-1 Bla, FIGS. 7 and 8), esterase, and hydrogen peroxide (H₂O₂), CDA generated approximately 1200-fold fluorescence turn-on (FIGS. 1b and 6b). In comparison, Amplex red showed a 413-fold turn-on in the presence of horseradish peroxidase (HRP) and H₂O₂ (FIGS. 6a and 6c).

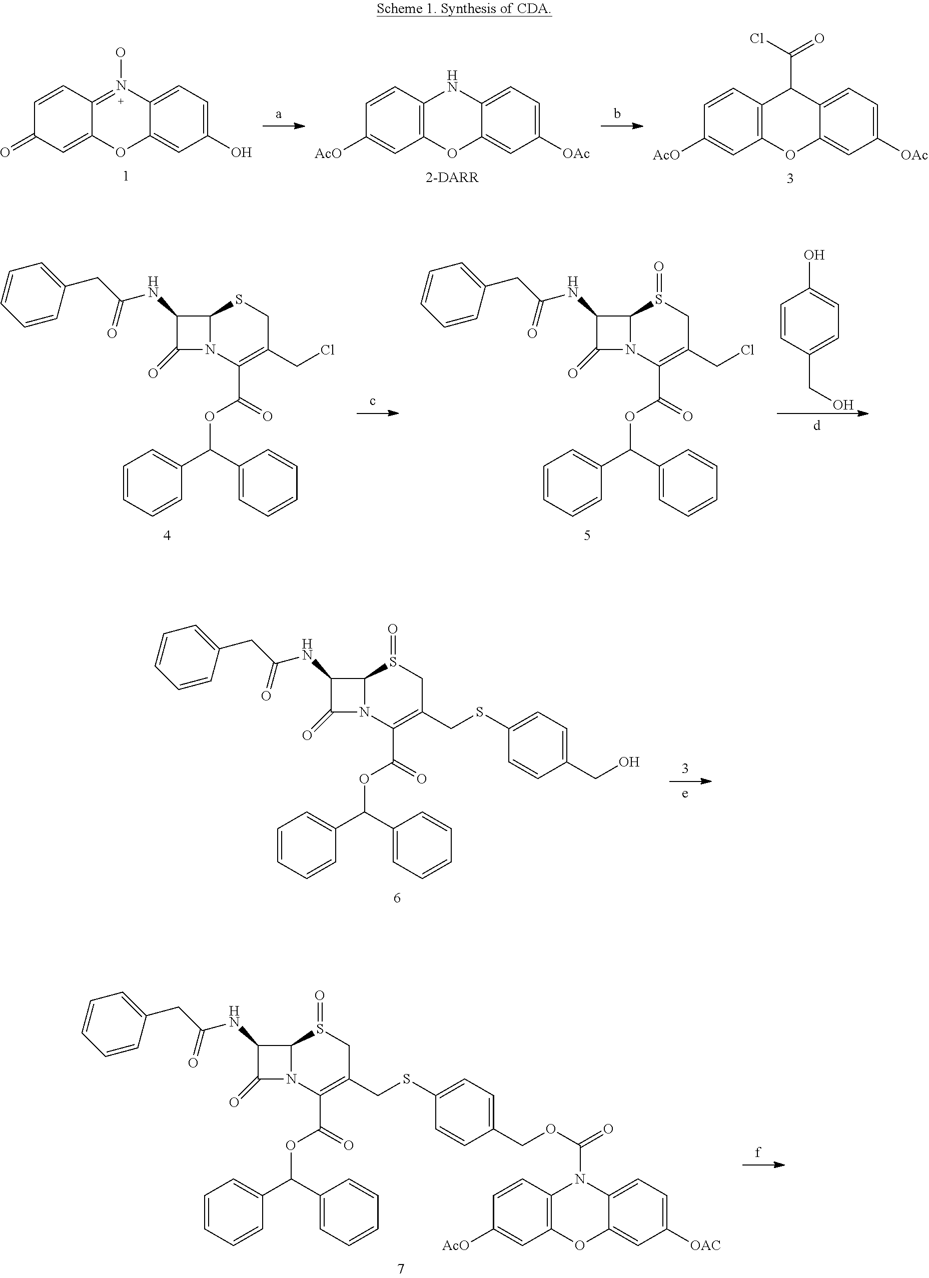

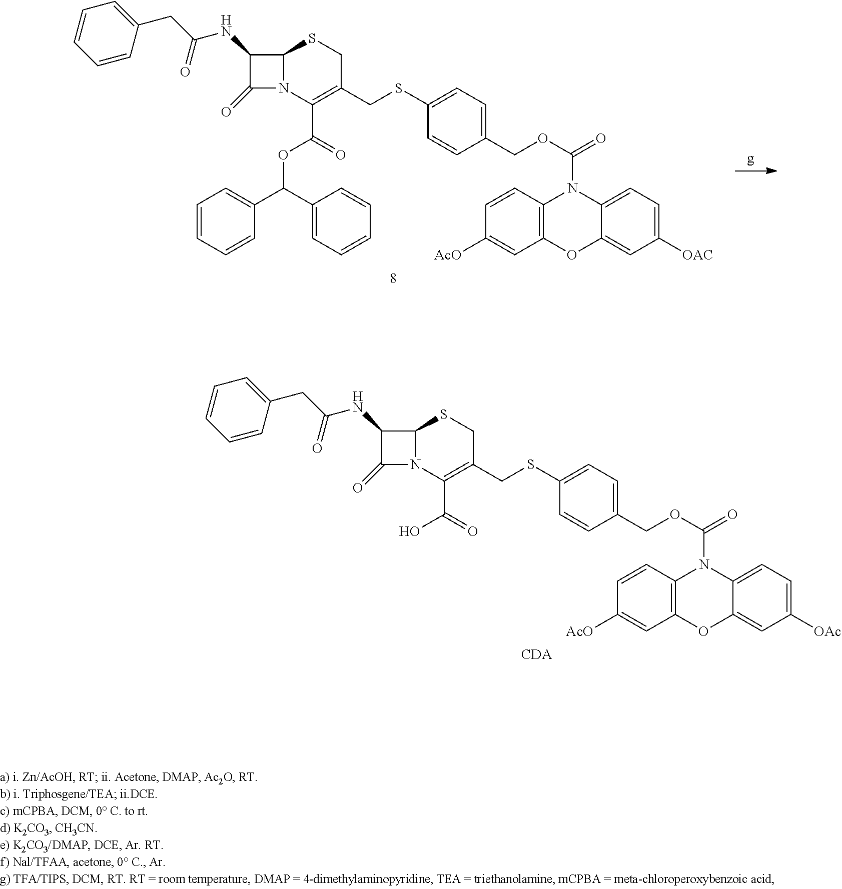

### Characterization of CDA With Recombinant β-Lactamases

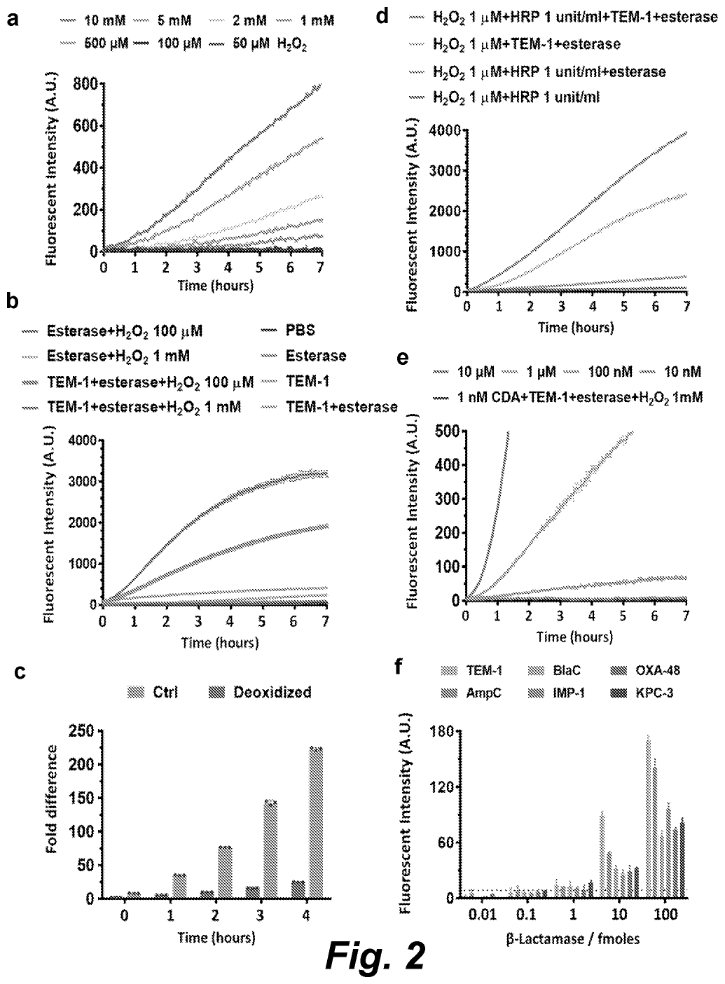

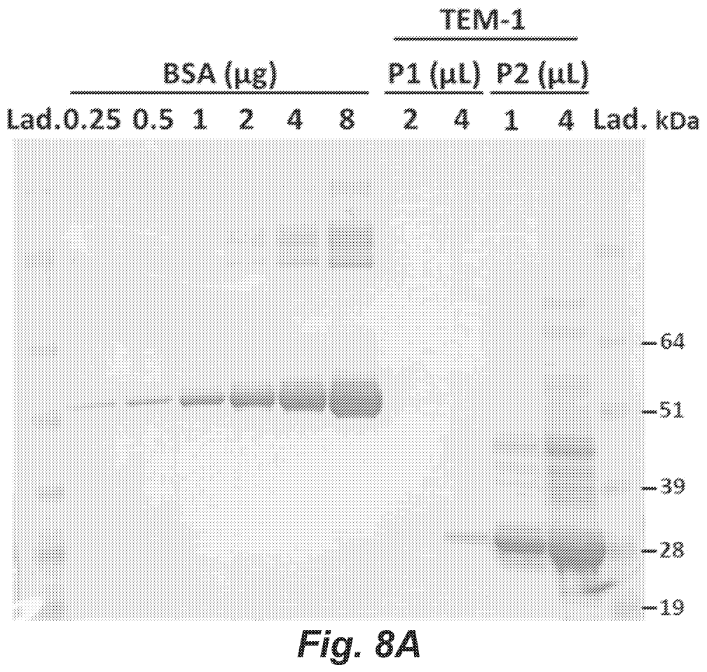

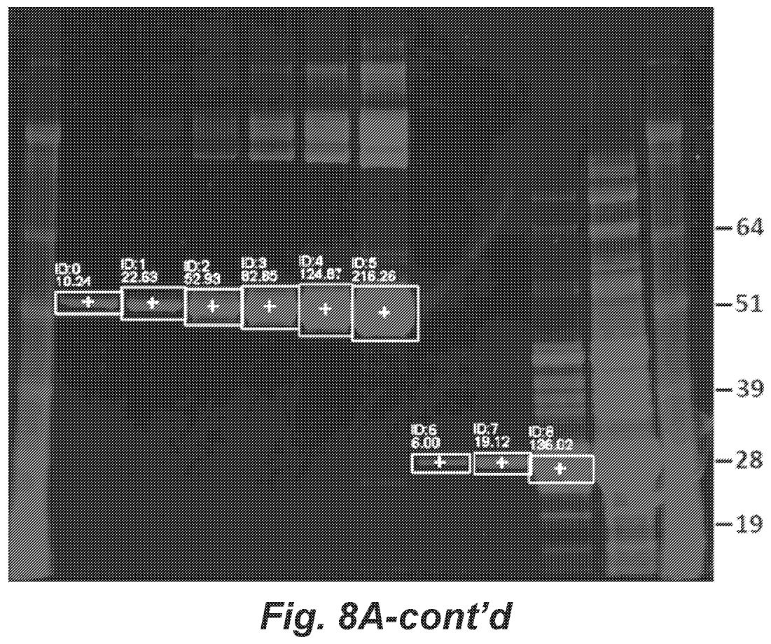

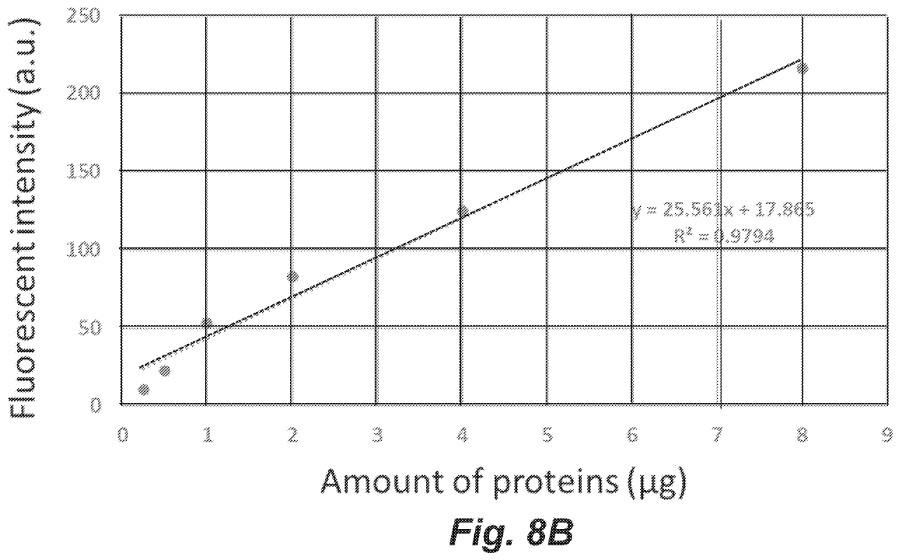

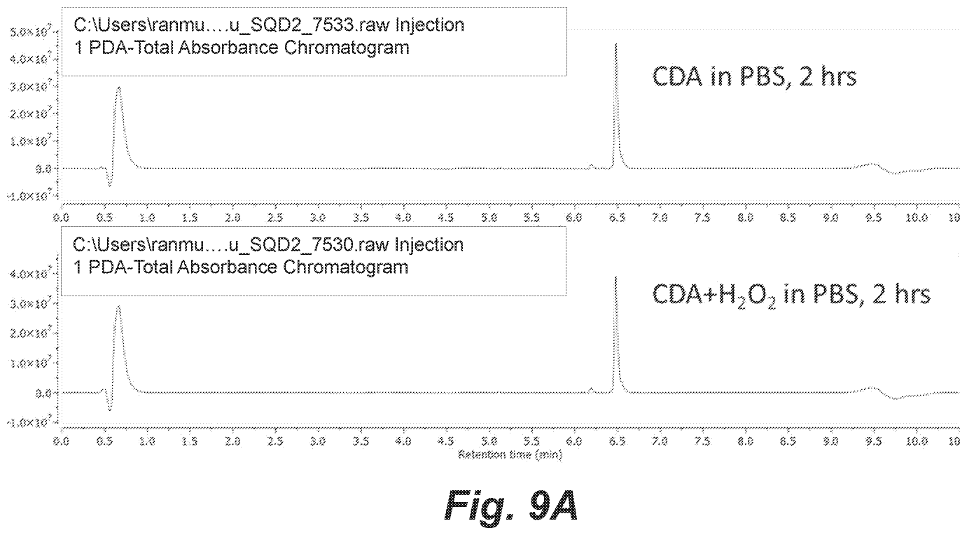

As oxidation is the essential step in converting β-lactamase/esterase processed CDA to resorufin, the stability of CDA was evaluated in different concentrations of H₂O₂ (FIG. 2a). Almost no spontaneous hydrolysis was noticed in the presence of up to 1 mM H₂O₂ in PBS within 2 hours of incubation at room temperature. The sulfur center of CDA was also stable in H₂O₂ (1 mM) (FIG. 9). DARR with two ester bonds presented similar concentration-dependent stability in H₂O₂ and fast response to esterase (FIGS. 10 and 11). CDA treated with TEM-1 Bla and esterase displayed fluorescence turn-on without H₂O₂ (FIG. 2b), because the dissolved oxygen in solution efficiently oxidized the TEM-1 Bla/esterase processed CDA into resorufin (FIG. 2c). Either H₂O₂ at a concentration above 1 mM alone or H₂O₂ (approximately 1 μM) with HRP (0.1-1 unit/mL) could significantly enhance the fluorescence turn-on (FIGS. 2d and 12).

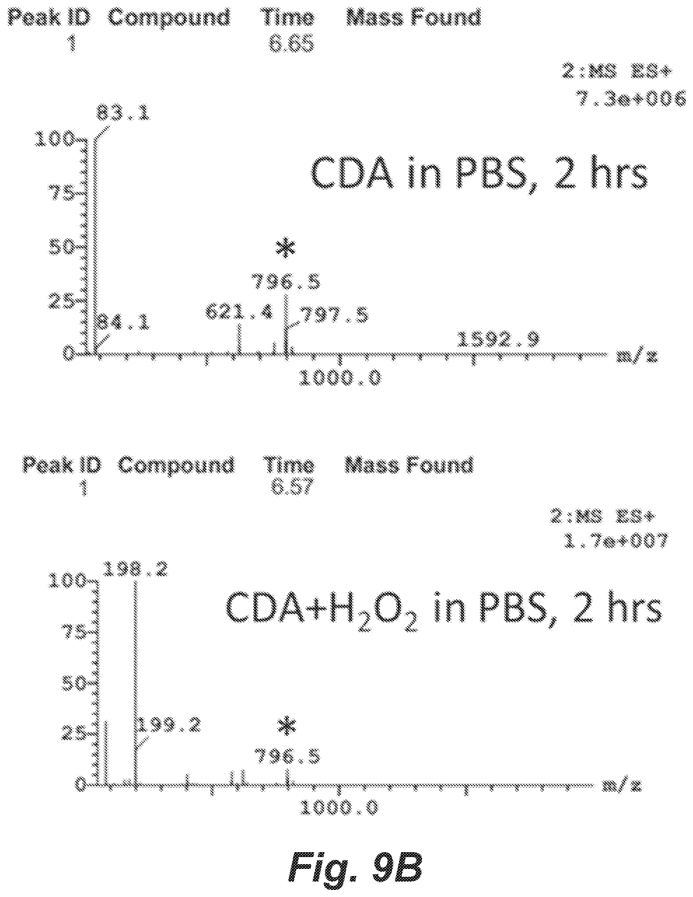

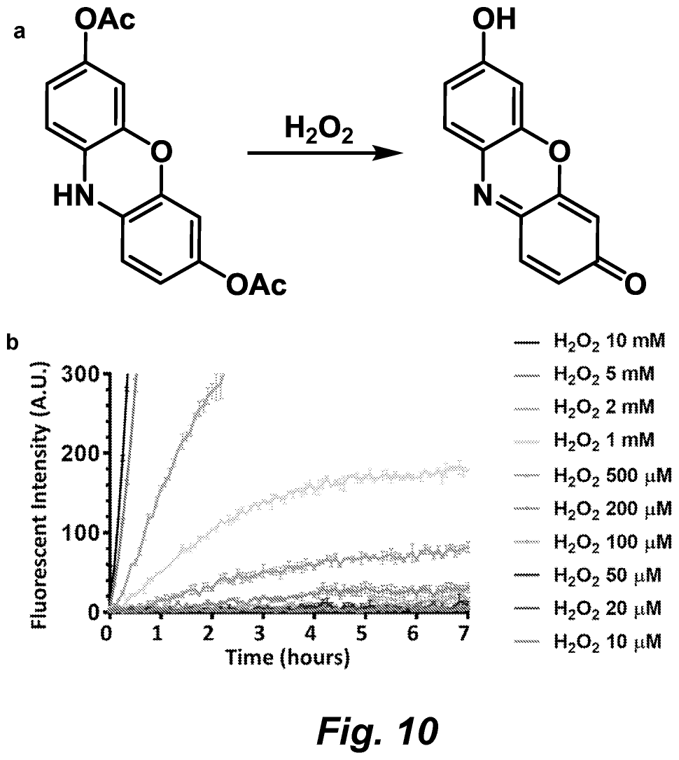

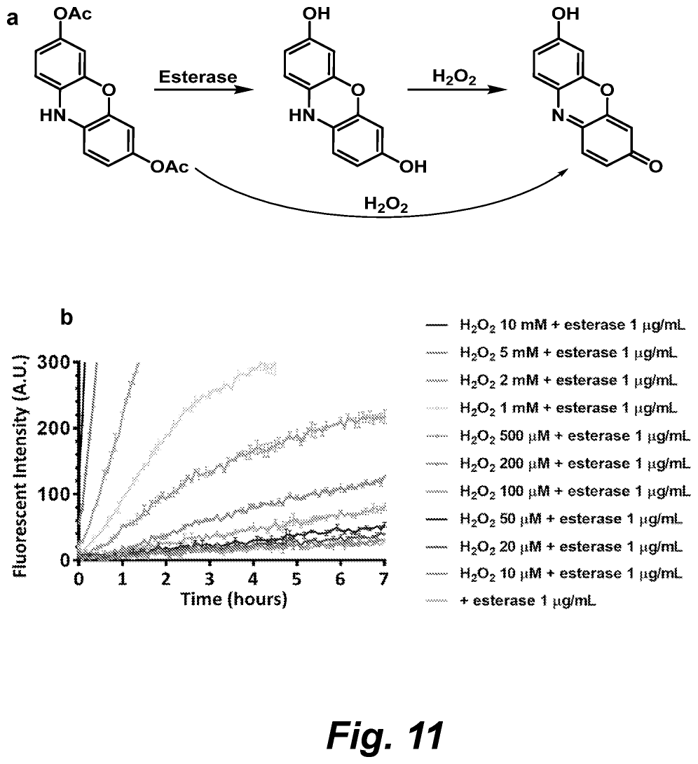

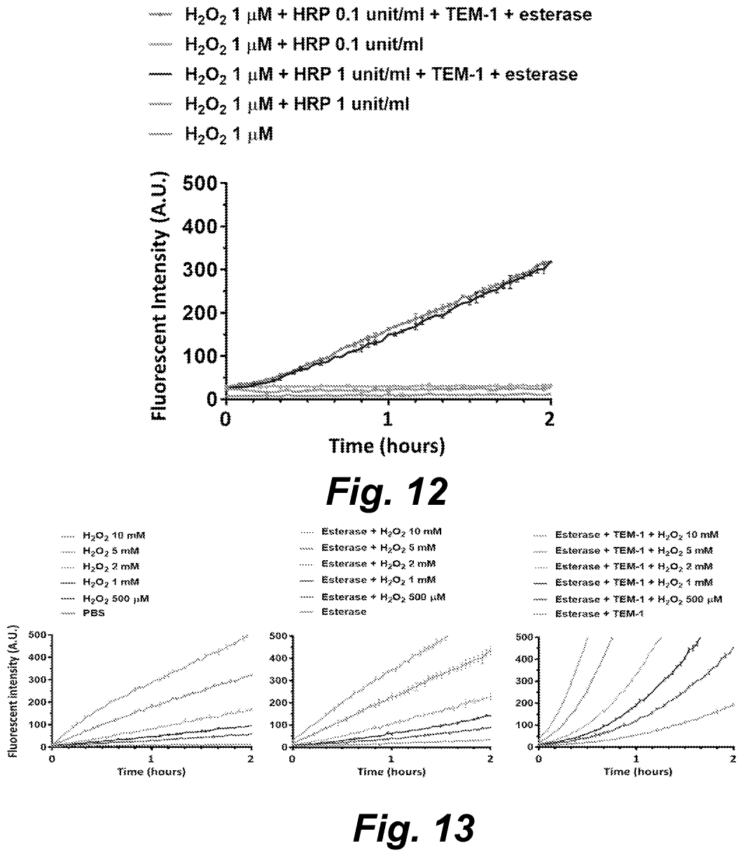

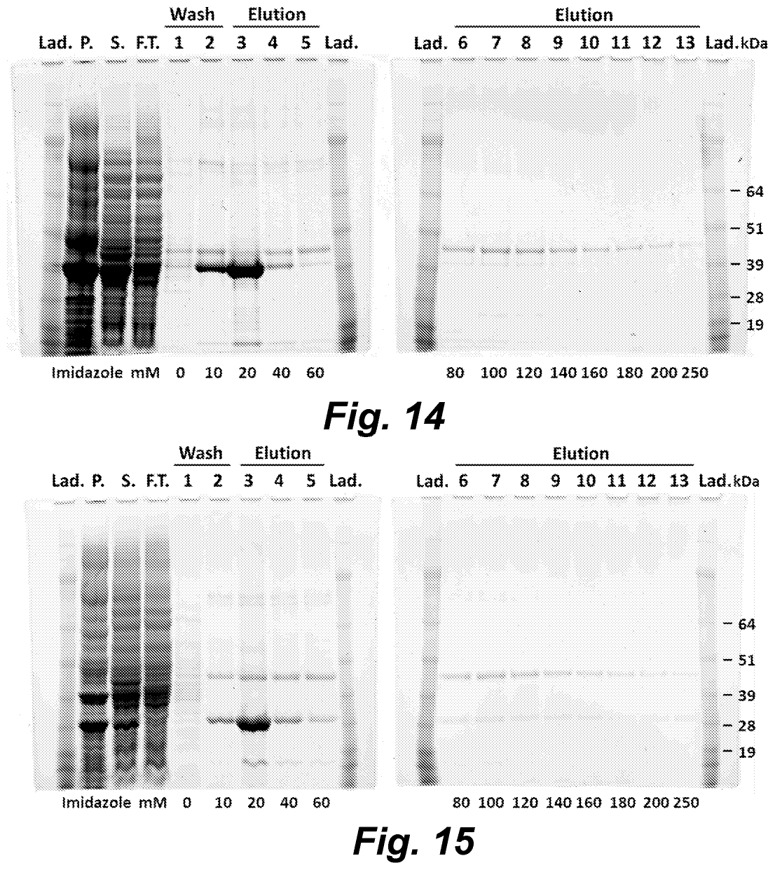

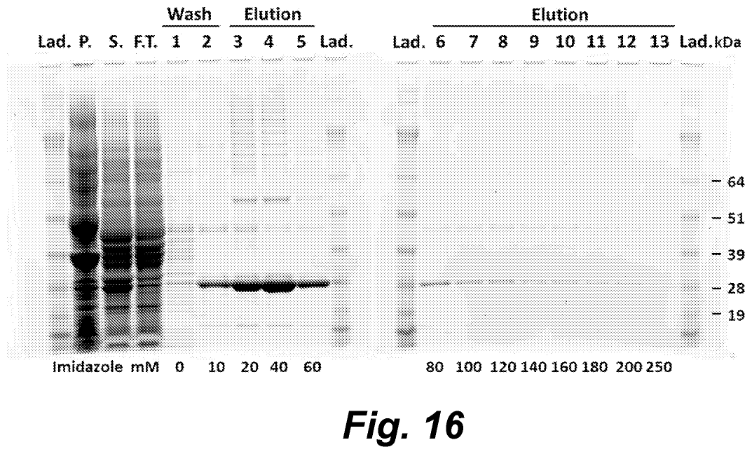

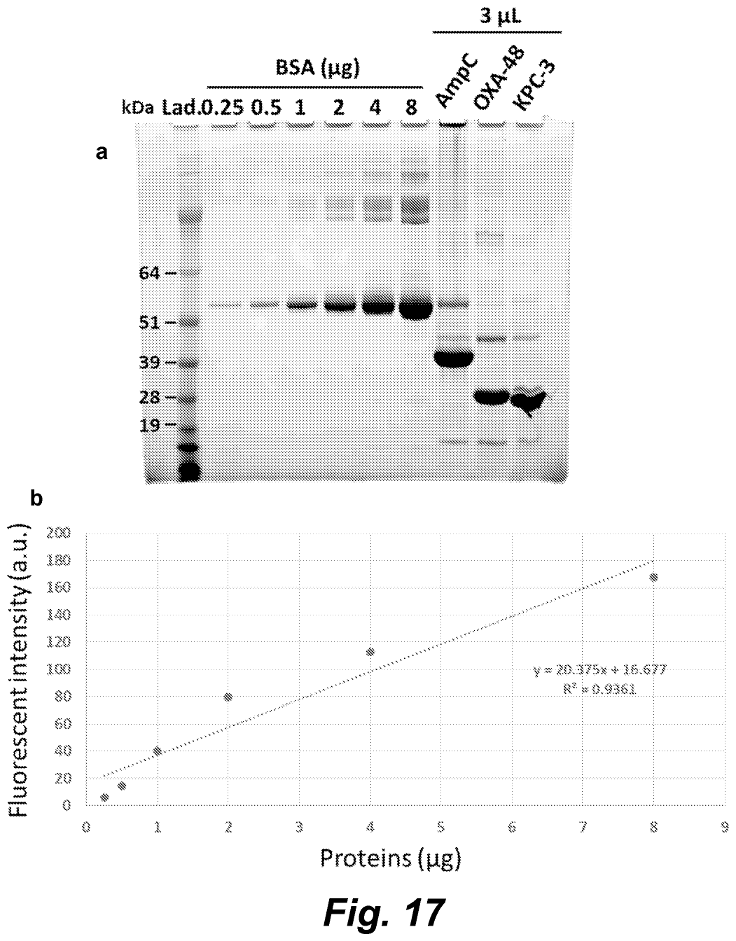

100 nM CDA at the minimum is required for reporting β-lactamase/esterase activity and 10 M CDA generated ideal signal strength (FIG. 2e). Esterases alone barely changed the response of CDA to H₂O₂ (FIG. 13). To demonstrate the ability of CDA in detecting a broad range of β-lactamases, several clinically prevalent β-lactamases were expressed and purified in addition to TEM-1 Bla over all four Ambler classes: extended spectrum AmpC (class C), Mycobacterium tuberculosis specific BlaC (class A), and carbapenemases IMP-1 (class B), OXA-48 (class D) and KPC-3 (class A) (FIGS. 14-19). CDA could detect all β-lactamases at as low as 1 femtomole in 2 hours at room temperature, a sensitivity unachievable with our previous broad spectrum β-lactamases or carbapenemases specific fluorogenic probes (FIG. 2f). Limits of detection here were determined by the fluorescent signal equaled to three times of the standard deviation of the negative controls: CDA in 1 mM H₂O₂/PBS with esterase at room temperature for 2 h. Together, these data suggested a good sensitivity and selectivity of CDA towards β-lactamases.

### Characterization of CDA With Bacteria

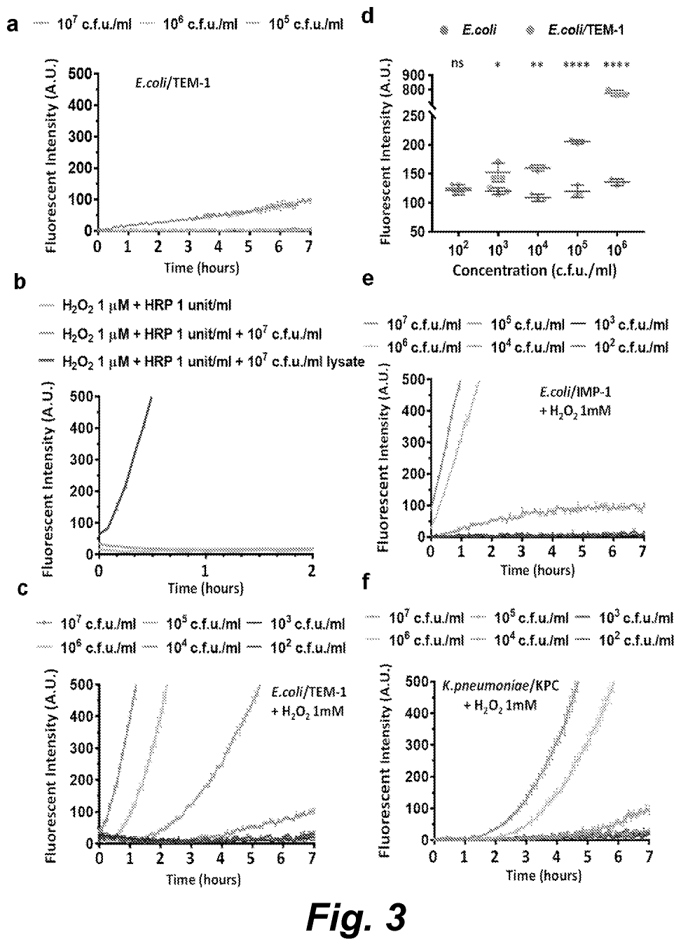

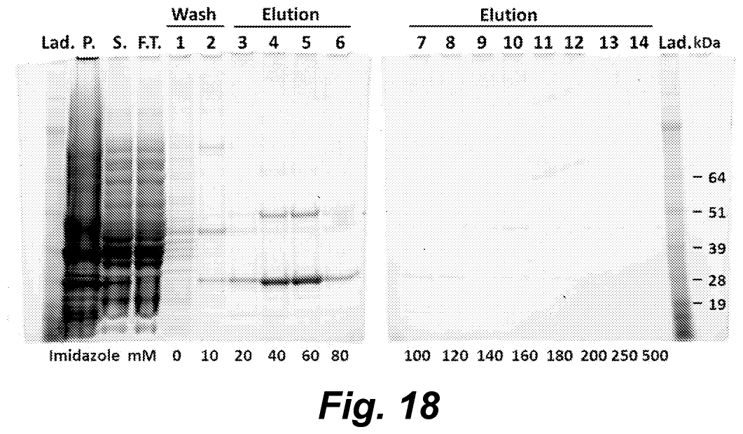

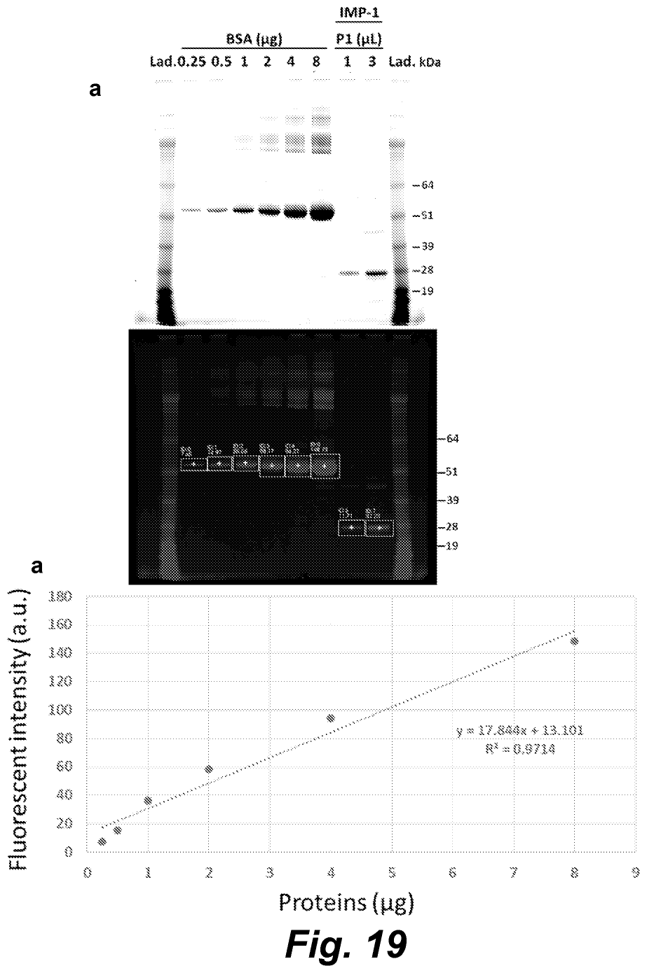

It was studied if CDA could report β-lactamase activity in live E. coli and Klebsiella pneumoniae (K. pneumoniae) which are commonly found in many infections. Three transformed E. coli strains expressing the most clinically prevalent β-lactamases including TEM-1 Bla (E. coli/TEM-1) and two carbapenemases were evaluated: a metallo-β-lactamase IMP-1 (E. coli/IMP-1) and a unique class A β-lactamase KPC-3 (E. coli/KPC-3) that hydrolyze carbapenem antibiotics. The number of bacteria in the assay was verified by calibrating the optical density measurement at 600 nm (OD600) and bacterial colony-forming unit (FIG. 20). Because CDA could be activated by TEM-1 Bla and esterase in PBS without extra oxidative reagents, we explored whether CDA alone could detect E. coli/TEM-1. As shown in FIG. 3a, to a limited extent, CDA became detectable only with a large number of TEM-1 Bla producing bacteria (107 c.f.u./mL) (FIG. 21a). This suggests the level of H₂O₂ or oxygen within bacteria may not be sufficient to oxidize the hydrolyzed product to resorufin. The addition of HRP and H₂O₂ (1 μM) contributed little to the fluorescence increase unless bacteria were lysed (FIG. 3b). As HRP has no means to penetrate cell membranes, these results suggested that the reduced resorufin was mostly retained intracellularly and 1 μM H₂O₂ was not efficient to oxidize it into resorufin.

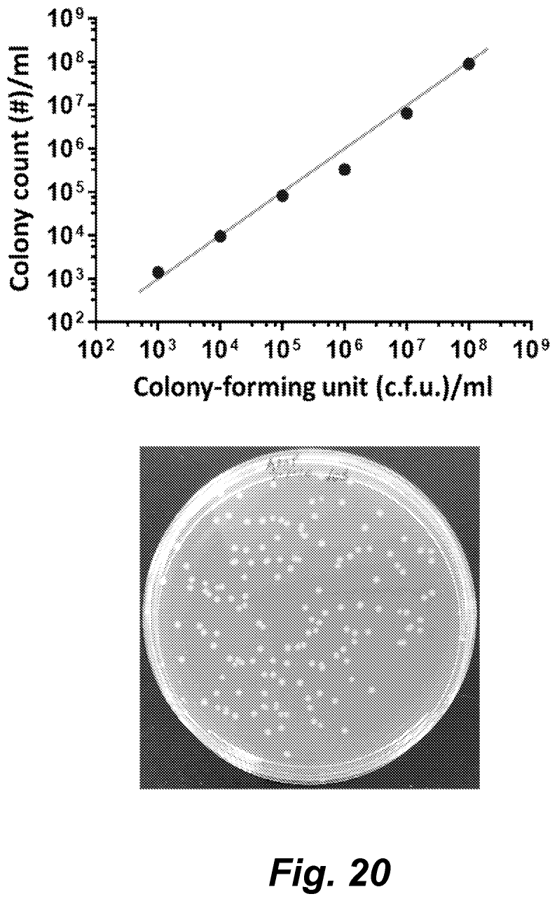

To address the question of whether a higher concentration of H₂O₂ would equilibrate across membranes of viable bacteria and accelerate the oxidation of processed CDA, a serial dilution of H₂O₂ (100 μM, 500 μM, 1 mM, 2 mM) was incubated with CDA and different concentrations of E. coli and E. coli/TEM-1 for a longitudinal reading (FIGS. 3c, 21b, and 22). Much-improved sensitivity was observed in 1 mM H₂O₂/CDA which detected 105 c.f.u./mL E. coli/TEM-1 within 2-3 hours and 104 c.f.u./mL after 4 hours incubation at room temperature. A longer preincubation (approximately 20 hours) with CDA before H₂O₂ addition could detect as low as 103 c.f.u./mL (FIG. 3d).

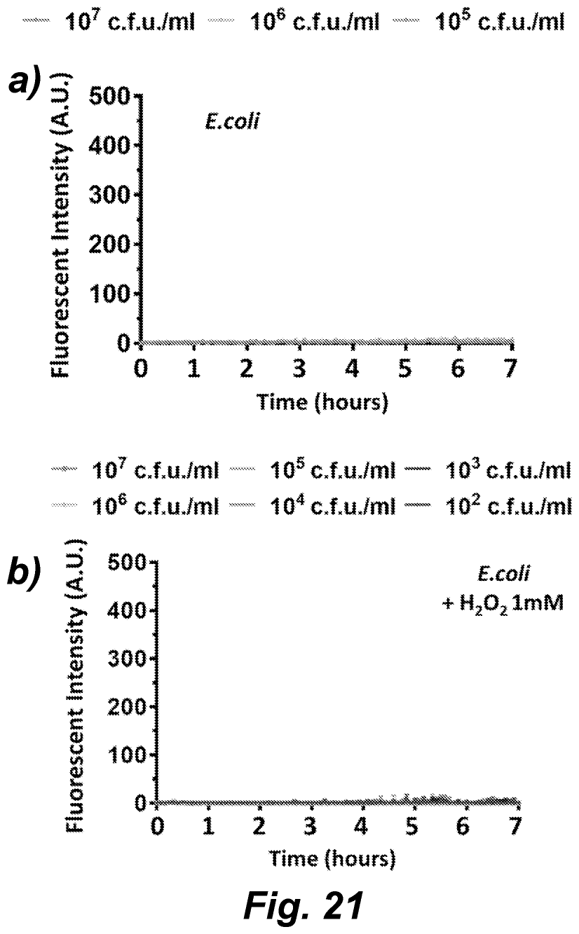

Factoring the 100 μL assay volume, as low as 100 bacteria in one 96 well were detectable with the assay. A concentration of H₂O₂ at 1-2 mM efficiently oxidized the processed CDA to resorufin inside E. coli. Next, the assay was used to detect E. coli/IMP-1 and E. coli/KPC-3 (FIGS. 3e and 23). E. coli/IMP-1 was detected at 105 c.f.u./mL within 2 hours, and E. coli/KPC-3 could be reliably detected at around 10⁵-10⁶ c.f.u./mL.

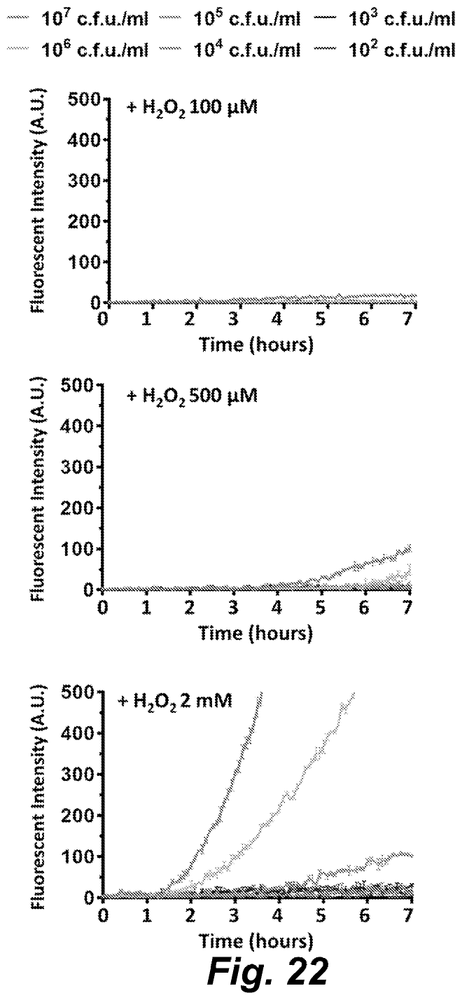

As KPC-3 carbapenemase has shown comparable activity to TEM-1 Bla in hydrolyzing cephalosporin substrates, it was investigated whether the expression level of β-lactamases contributed to the variation in detection sensitivity. When TEM-1 Bla transformed E. coli were not induced (FIG. 24), the fluorescence intensity was compromised due to the low expression of TEM-1 Bla, but 10⁶-10⁷ c.f.u./mL bacteria was still detectable within 2-3 hours.

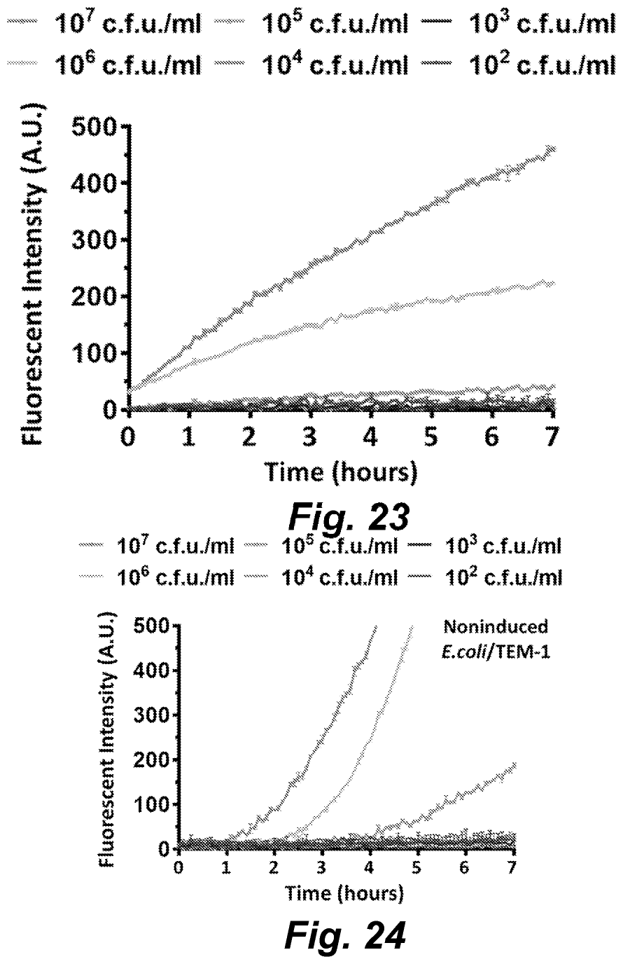

CDA was also tested for detecting K. pneumoniae. Two clinical isolates, either imipenem sensitive or resistant by expressing a KPC carbapenemase (K. pneumoniae/KPC) were incubated with CDA and H₂O₂ (FIGS. 3f and 25). The number of K. pneumoniae in the assay was also verified and OD600 of 1.0 gave roughly 1×10⁹ c.f.u./mL bacteria. Similar to E. coli/KPC-3, 10⁶ c.f.u./mL KPC-positive K. pneumoniae could be detected within 2-3 hours. Collectively, these data suggested that the CDA/H₂O₂ assay could be useful for detecting cephalosporinase- and carbapenemase-expressing bacteria in clinic.

### Development of a Rapid Fluorogenic Assay for Screening Lactam Antibiotics Resistant Urinary Tract Infections

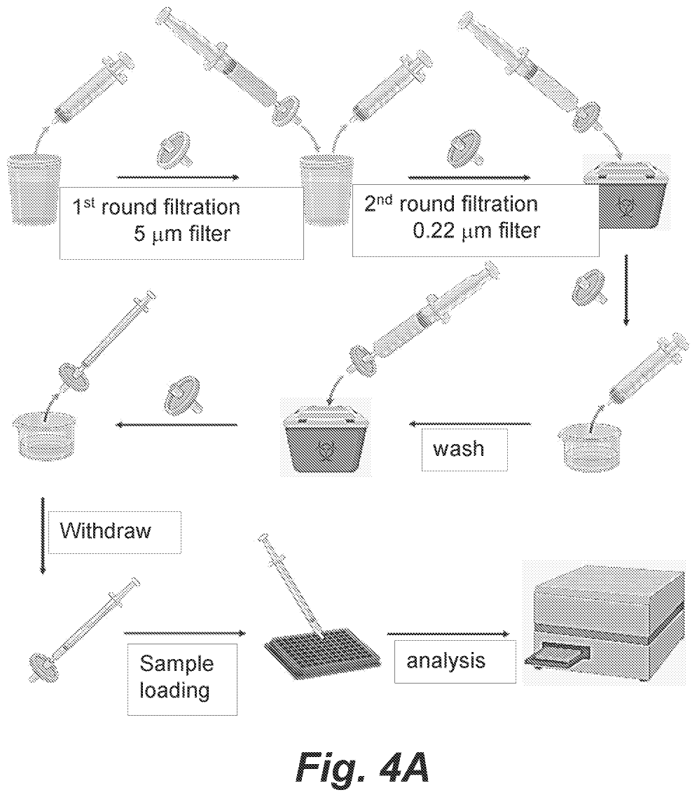

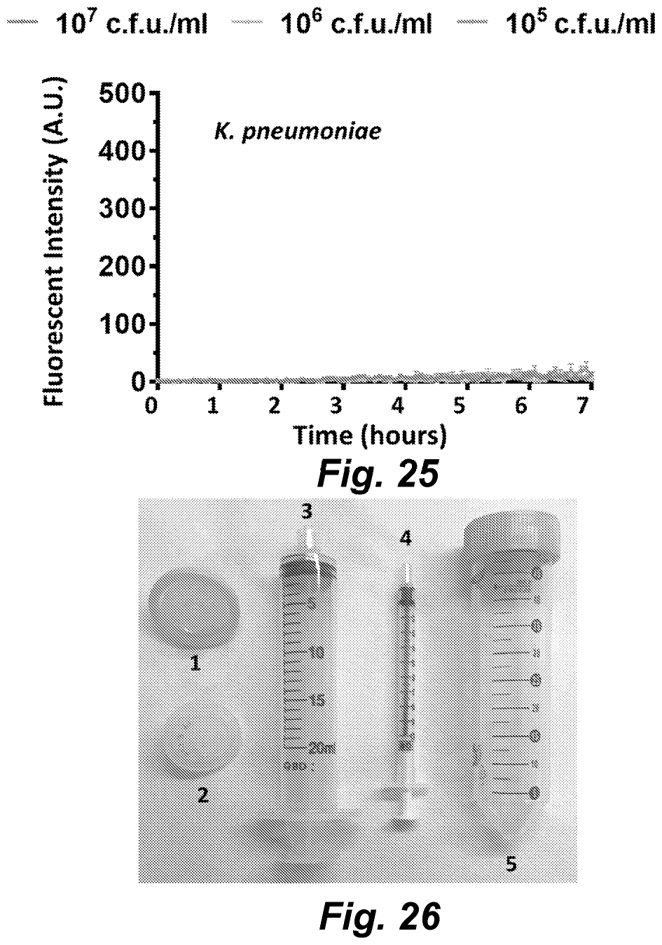

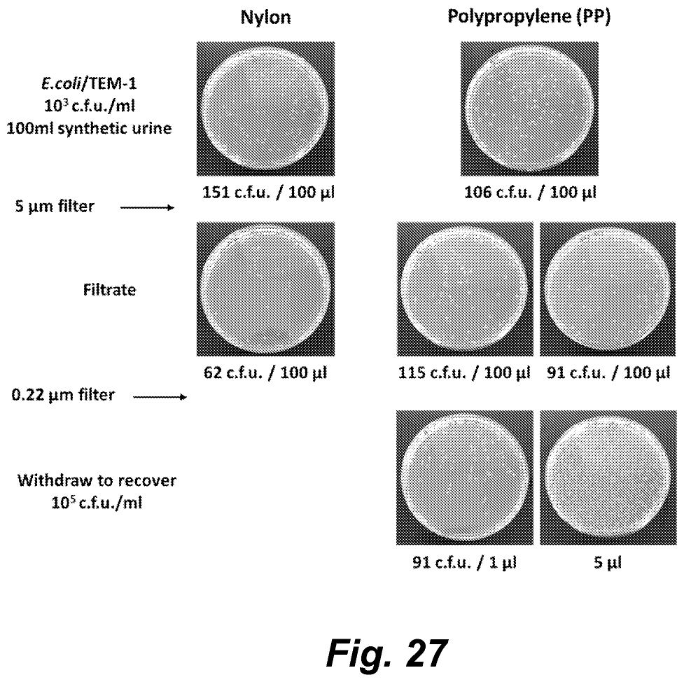

To develop an easy-to-use, point-of-care assay for screening cephalosporin and carbapenem resistance, a simple two-step filtration method was employed (FIG. 26) to prepare and concentrate pathogenic bacteria, similar to a procedure applied for improving culture-based AST previously (Avesar et al., Procs. Nat. Acad. Sci. U.S.A, 2017, 114, E5787-E5795). As shown in FIG. 4a, either processed or unprocessed patient samples can be filtrated sequentially through a 5-micron and a 0.22-micron syringe filter. The 5-micron filter would trap particles including cells and large debris, and bacteria would be collected by the 0.22-micron filter and washed by PBS. A reverse elution with PBS containing 1 mM H₂O₂ allowed immediate high-throughput analysis by a plate reader in a 96-well plate precoated with CDA. Synthetic urine samples were used as a model to test the workflow. 50 mL of synthetic urine spiked with bacteria could be concentrated to approximately 200 μL ready-to-use suspension within a few minutes; polypropylene filters recover bacteria more efficiently (>90%) than commonly used nylon filters (FIG. 27).

According to the protocol for urinary tract infection (UTI) events updated on January 2020 by CDC (Centers for Disease Control and Prevention, US), all symptomatic, catheter-associated symptomatic, and asymptomatic bacteremic UTI criteria require a positive urine culture with no more than 2 species of microorganisms, at least one of which is ≥10⁵ c.f.u./mL. Some clinical laboratories have set a lower threshold (10³-10⁴ c.f.u./mL) for reporting UTI under certain circumstances.

Accordingly, samples we prepared (approximately 50 mL each) containing 10³, 10⁴, and 10⁵ c.f.u./mL of engineered or clinically isolated bacteria confirmed with or without β-lactamases, including E. coli, K. pneumoniae, Enterobacter cloacae (E. cloacae) and Serratia marcescens (S. marcescens). As shown in FIG. 4b, after 250-fold enrichment, a time dependent increase of fluorescent signal was observed in all β-lactamase producing bacteria.

As negative controls, E. coli generated slightly increased signal while the K. pneumoniae was almost undetectable. the 3 times of the standard deviation of the mean fluorescent intensity of E. coli at different time points was used to predict the limit of detection: 10³ and 10⁴ c.f.u./mL of all β-lactamase producing bacteria were detectable within 2 hours incubation; 10⁵ c.f.u./mL could be detected within 1 hour. The fluorescent signals from E. coli expressing recombinant β-lactamases were constantly higher than those clinical isolates at 10⁴-10⁵ c.f.u./mL, which indicated a more robust β-lactamases expression in these engineered strains.

### Synthesis of CDA2 for Carbapenemases Detection

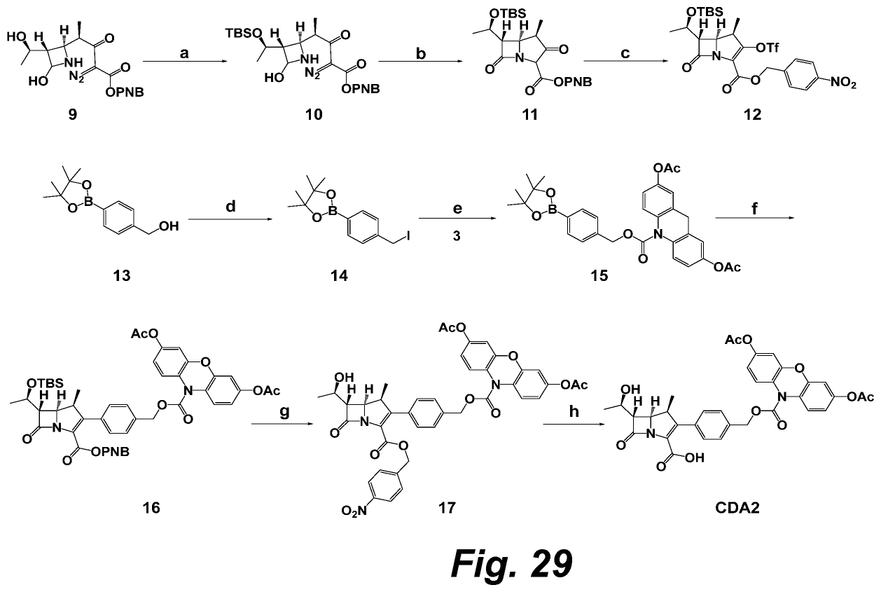

The cephalosporin moiety in CDA was replaced with a carbapenem and afforded a carbapenemase-specific probe CDA2 (Scheme 2, FIG. 29). We anticipate that CDA2 could only be activated by carbapenemase expressing pathogens such as carbapenem-resistant Enterobacteriaceae (CRE), and further hydrolyzed and oxidized to highly fluorescent resorufin. CDA2 will be utilized for detection of a serial of carbapenemase expressing bacteria and provide additional diagnostic value of the CDA assay in identification of life-threatening carbapenem antibiotics resistant infections in a point-of-care manner.

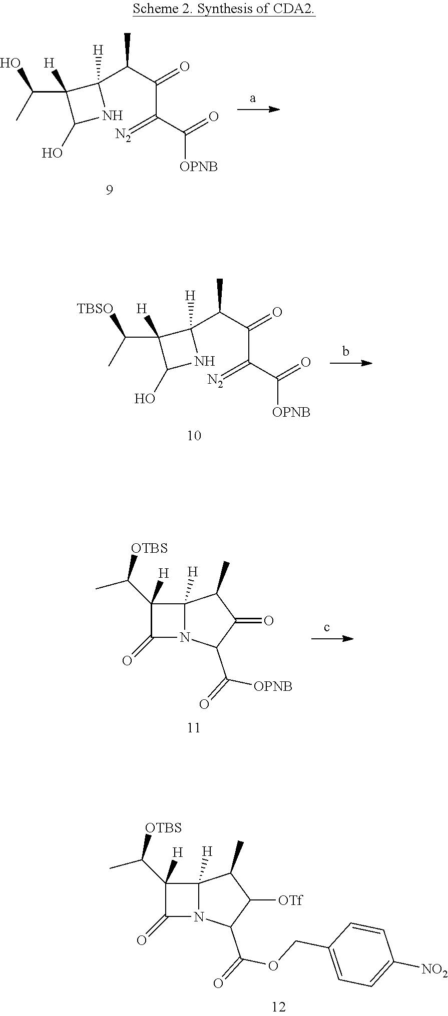

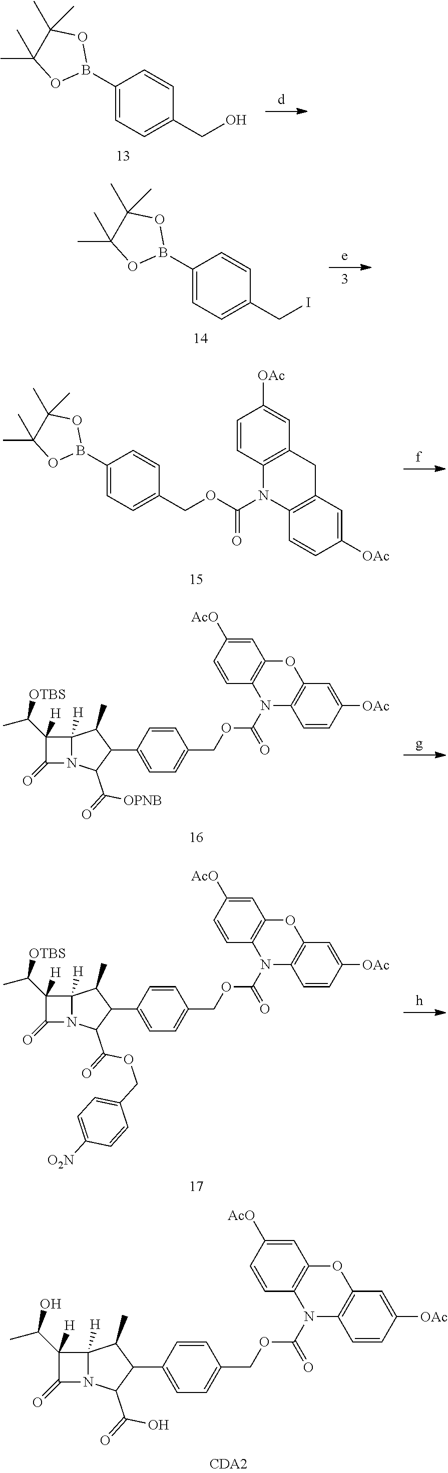

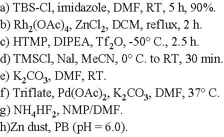

Accordingly, one aspect of the disclosure encompasses embodiments of a dual-caged cleavable probe comprising a β-lactamase- or carbapenemase-cleavable moiety conjugated to a self-immolative linker, wherein the self-immolative linker is conjugated to a fluorescently detectable label.

In some embodiments of this aspect of the disclosure, the probe can have the formula:
R₁-β-lactam-self-immolative linker-fluorescently detectable label, wherein R₁ can be an H, a halogen, a substituent selected from NO₂, CN, carboxyl, OH, an amine, a substituted or unsubstituted alkyl, a substituted or unsubstituted aryl, a substituted or unsubstituted cycloalkyl, a substituted or unsubstituted cycloalkenyl, a substituted or unsubstituted aryl, a substituted or unsubstituted biaryl, a substituted or unsubstituted fused aryl, a substituted or unsubstituted alkenyl, or a substituted or unsubstituted alkynyl; the β-lactam can be a cephalosporin or a carbapenem, wherein the cephalosporin comprises the structure (a) or (b) and the carbapenem can have the structure (c)

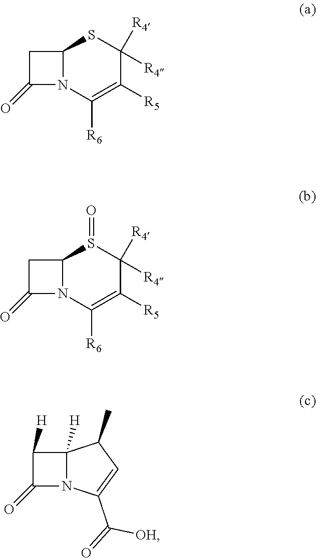

wherein R_{4′}, R_{4″}, R₅, and R₆ can be each independently a H, a halogen, a substituent selected from NO₂, CN, carboxyl, OH, an amine, a substituted or unsubstituted alkyl, a substituted or unsubstituted aryl, a substituted or unsubstituted cycloalkyl, a substituted or unsubstituted cycloalkenyl, a substituted or unsubstituted aryl, a substituted or unsubstituted biaryl, a substituted or unsubstituted fused aryl, a substituted or unsubstituted alkenyl, or a substituted or unsubstituted alkynyl; the self-immolative linker can have the structure:

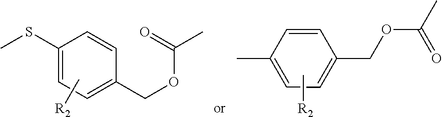

wherein R₂, can be a H, a halogen, a substituent selected from NO₂, CN, carboxyl, OH, an amine, a substituted or unsubstituted alkyl, a substituted or unsubstituted aryl, a substituted or unsubstituted cycloalkyl, a substituted or unsubstituted cycloalkenyl, a substituted or unsubstituted aryl, a substituted or unsubstituted biaryl, a substituted or unsubstituted fused aryl, a substituted or unsubstituted alkenyl, or a substituted or unsubstituted alkynyl. In some embodiments of this aspect of the disclosure, the fluorescently detectable label can have the structure:

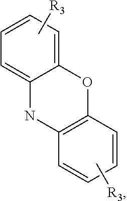

wherein R₃ can be a hydrogen, a halogen, a substituent selected from NO₂, CN, carboxyl, OH, an amine, a substituted or unsubstituted alkyl, a substituted or unsubstituted aryl, a substituted or unsubstituted cycloalkyl, a substituted or unsubstituted cycloalkenyl, a substituted or unsubstituted aryl, a substituted or unsubstituted biaryl, a substituted or unsubstituted fused aryl, a substituted or unsubstituted alkenyl, or a substituted or unsubstituted alkynyl.

In some embodiments of this aspect of the disclosure, the carbapenem can be selected from the group consisting of biapenem, ertapenem, doripenem, imipenem, and panipenem:

In some embodiments of this aspect of the disclosure, the probe can be a dual-caged N-cephalosporin-caged 3,7-diesterphenoxazine beta-lactamase-cleavable probe or a dual-caged carbapenem-caged 3,7-diesterphenoxazine carbapenemase-cleavable probe. In some embodiments of this aspect of the disclosure, the dual-caged N-cephalosporin-caged 3,7-diesterphenoxazine beta-lactamase-cleavable probe has the structure of formula I:

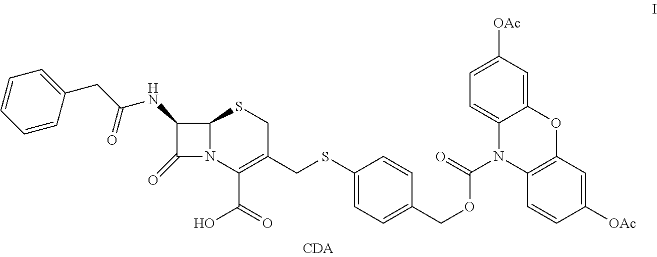 [SMILES: CC(=O)Oc1ccc2c(c1)Oc1cc(OC(C)=O)ccc1N2C(=O)OCc1ccc(SCC2=C(C(=O)O)N3C(=O)[C@@H](NC(=O)Cc4ccccc4)[C@H]3SC2)cc1]

In some embodiments of this aspect of the disclosure, the dual-caged carbapenem-caged 3,7-diesterphenoxazine carbapenemase-cleavable probe can have the structure of formula II:

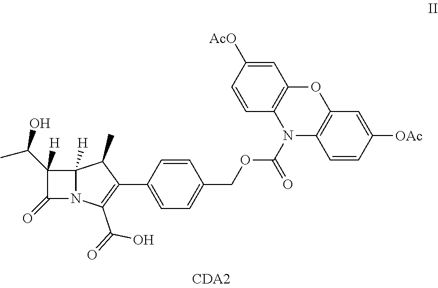 [SMILES: CC(=O)Oc1ccc2c(c1)Oc1cc(OC(C)=O)ccc1N2C(=O)OCc1ccc(C2=C(C(=O)O)N3C(=O)[C@H]([C@@H](C)O)[C@H]3[C@H]2C)cc1]

In some embodiments of this aspect of the disclosure, the detectable label is 10H-phenoxazine-3,7-diyl diacetate (2-DARR).

Another aspect of the disclosure encompasses embodiments of a method of detecting the presence of a bacterial strain that has resistance to at least one of a beta-lactam antibiotic or a carbapenem antibiotic, wherein said method comprises contacting a population of bacteria suspected of being resistant to at least one of a beta-lactam antibiotic or a carbapenem antibiotic with a dual-caged N-cephalosporin-caged 3,7-diesterphenoxazine beta-lactamase-cleavable probe or a dual-caged carbapenem-caged 3,7-diesterphenoxazine carbapenem-cleavable probe in the presence of an oxidizing agent, and measuring a fluorescent signal, wherein a detectable fluorescent signal indicates that the bacterial strain has at least one of a beta-lactamase or a carbapenemase.

In some embodiments of this aspect of the disclosure, the dual-caged N-cephalosporin-caged 3,7-diesterphenoxazine beta-lactamase-cleavable probe or a dual-caged carbapenem-caged 3,7-diesterphenoxazine carbapenem-cleavable probe can comprise a β-lactamase- or carbapenemase-cleavable moiety conjugated to a self-immolative linker, wherein the self-immolative linker is conjugated to a fluorescently-detectable label moiety.

In some embodiments of this aspect of the disclosure, the fluorescently detectable label moiety is 10H-phenoxazine-3,7-diyl diacetate (2-DARR).

In some embodiments of this aspect of the disclosure, the dual-caged N-cephalosporin-caged 3,7-diesterphenoxazine beta-lactamase-cleavable probe has the structure of formula I:

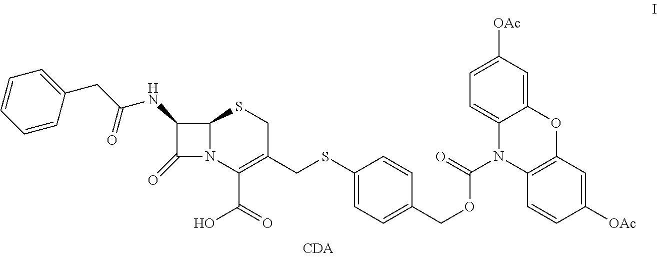 [SMILES: CC(=O)Oc1ccc2c(c1)Oc1cc(OC(C)=O)ccc1N2C(=O)OCc1ccc(SCC2=C(C(=O)O)N3C(=O)[C@@H](NC(=O)Cc4ccccc4)[C@H]3SC2)cc1]

and the dual-caged N-cephalosporin-caged 3,7-diesterphenoxazine beta-lactamase-cleavable probe has the structure of formula II:

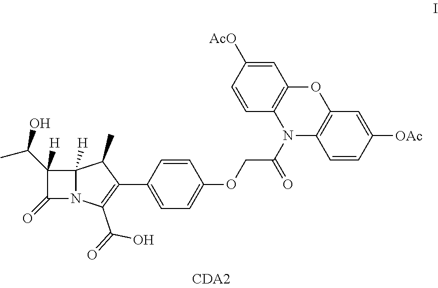 [SMILES: CC(=O)Oc1ccc2c(c1)Oc1cc(OC(C)=O)ccc1N2C(=O)COc1ccc(C2=C(C(=O)O)N3C(=O)[C@H]([C@@H](C)O)[C@H]3[C@H]2C)cc1]

In some embodiments of this aspect of the disclosure, the oxidizing agent is hydrogen peroxide.

In some embodiments of this aspect of the disclosure, the method can further comprise culturing a biological sample from a human or animal to generate a population of bacteria from the sample and concentrating the population of cultured bacteria.

Yet another aspect of the disclosure encompasses embodiments of a method of synthesis of a dual-caged N-cephalosporin-caged 3,7-diesterphenoxazine beta-lactamase-cleavable probe having the structure of formula I:

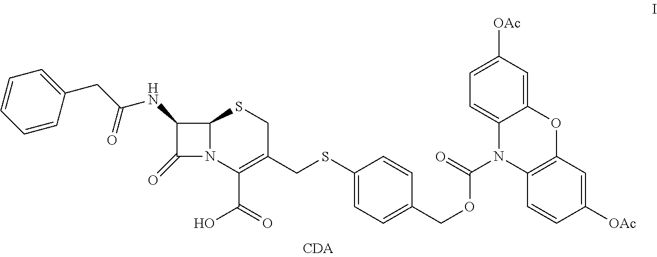 [SMILES: CC(=O)Oc1ccc2c(c1)Oc1cc(OC(C)=O)ccc1N2C(=O)OCc1ccc(SCC2=C(C(=O)O)N3C(=O)[C@@H](NC(=O)Cc4ccccc4)[C@H]3SC2)cc1]

wherein the method is according to Scheme 1 (FIG. 28) of the disclosure.

Still another aspect of the disclosure encompasses embodiments of a method of synthesis of a dual-caged N-cephalosporin-caged 3,7-diesterphenoxazine beta-lactamase-cleavable probe has the structure of formula II:

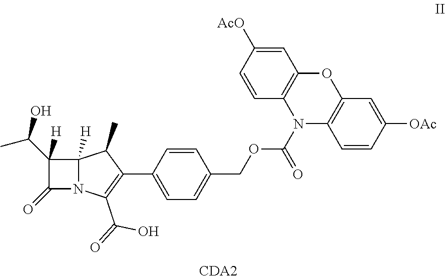 [SMILES: CC(=O)Oc1ccc2c(c1)Oc1cc(OC(C)=O)ccc1N2C(=O)OCc1ccc(C2=C(C(=O)O)N3C(=O)[C@H]([C@@H](C)O)[C@H]3[C@H]2C)cc1]

wherein the method is according to Scheme 2 (FIG. 29) of the disclosure.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

### Example 1

Materials and General Methods: All chemicals were purchased from commercial sources as specified below. Analytical TLC was performed with 0.25 mm silica gel 60F plates with fluorescent indicator (254 nm). The ¹H and ¹³C NMR spectra were taken on Varian 400 MHz, 500 MHz or 600 MHz magnetic resonance spectrometer. Data for ¹H NMR spectra are reported as follows: chemical shifts are reported as o in units of parts per million (ppm) relative to tetramethylsilane (δ=0, s); multiplicities are reported as follows: s (singlet), d (doublet), t (triplet), q (quartet), dd (doublet of doublets), m (multiplet), or br (broadened); coupling constants are reported as a J value in Hertz (Hz); the number of protons (n) for a given resonance is indicated nH, and based on the spectral integration values. HPLC was performed on a Dionex HPLC System (Dionex Corporation) equipped with a GP50 gradient pump and an inline diode array UV-Vis detector. A reversed-phase C18 (Phenomenax, 5 μm, 10×250 mm or Dionex, 5 μm, 4.6×250 mm) column was used with a MeCN (B)/H₂O (A) gradient mobile phase containing 0.1% trifluoroacetic acid at a flow of 1 or 3 mL/min for the analysis.

### Example 2

Preparation of Compound 10H-phenoxazine-3,7-diyl diacetate (2-DARR):

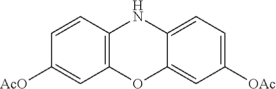 [SMILES: CC(=O)Oc1ccc2c(c1)Oc1cc(OC(C)=O)ccc1N2]

Compound 2-DARR was prepared according to a method previously reported. Briefly, step 1: To a 500 mL round-bottom flask and argon sparged, a solution of zinc powder (1.2 g, 20 mmol), resazurin sodium salt (compound 1, 1 g, 4.0 mmol), and glacial acetic acid (30 mL) were added and stirred vigorously at room temperature for 4 h. After the reacting solvent was evaporated under reduced pressure, a brownish crude product was obtained and used without purifying for next step. Step 2: Under the argon atmosphere, to the same 500 mL round-bottom flask, a stirred solution of the previous crude product in acetone (30 mL) was added N, N-dimethyl-4-aminopyridine (350 mg, 2.1 mmol) in a portion and added dropwise acetic anhydride (1.0 mL, 10.0 mmol). The mixture was stirred and monitored by TLC for around 6 hours. Zinc powder was removed by Celite filtration, and the filtrate mixture was evaporated under reduced pressure to afford a crude product. Flash column with EtOAc: hexane=1:5 to 1:1 gave a yellow crude product in 420 mg.

### Example 3

Preparation of Compound 10-(chlorocarbonyl)-10H-phenoxazine-3,7-diyl diacetate (3).

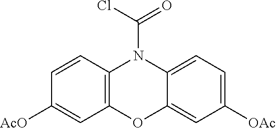 [SMILES: CC(=O)Oc1ccc2c(c1)Oc1cc(OC(C)=O)ccc1N2C(=O)Cl]

Under argon protection, to an ice-bathed 100 mL flask, a solution of 3,7-diacetoxyphenoxazine (compound 2) (300 mg, 1.0 mmol) and TEA (0.32 mL, 2.2 mmol) in mL dichloroethane was stirred for 15 min, then it was added dropwise a solution of triphosgene (1.35 g, 4.5 mmol) in DCE (10 mL) at 0° C. The reaction mixture was stirred at room temperature under an argon atmosphere for 5 h with HPLC monitoring. Then the mixture was diluted with DCM (60mL) and the resulting mixture was extracted with water (60 mL) and brine (60 mL) three times. The organic layer was extracted and dried with anhydrous Na₂SO₄. A white solid was obtained by silica-gel column chromatography with the eluent of EtOAc: hexane=1:4 to 1:1 in 49% yield (181 mg). ¹H NMR (400 MHz, CDCl₃) δ 7.59 (d, J=8.8 Hz, 2H), 6.97-6.95 (m, 2H), 6.92 (dd, J=8.8, 2.5 Hz, 2H), 2.31 (s, 6H); HRMS: Calculated for C₁₇H₁₂CINNaO₆ ([M+Na]⁺):

384.0245; Found: 384.0241.

### Example 4

Preparation of Compound Benzhydryl 3-(chloromethyl)-8-oxo-7-(2-phenylacetamido)-5-thia- 1-azabicyclo[4.2.0]oct-2-ene-2-carboxylate 5-oxide (5).

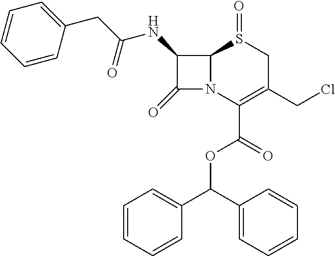

A solution of the cephalosporin chloride precursor (4, purchased from Pharmacore, China, 533 mg, 1.0 mmol) in anhydrous DCM was cooled to 0° C. prior to the addition of meta-chloroperoxybenzoic acid (172 mg, 1.0 mmol). The reaction was stirred at 0° C. for 30 min (a white precipitate was formed during this time) and subsequently for another 1 hour at room temperature. Silicon column purification afforded pure compound 5 (550 mg, 98%).

¹H NMR (500 MHz, DMSO) δ 8.48 (d, J=8.3 Hz, 1H), 7.53 (d, J=7.2 Hz, 2H), 7.44 (d, J=7.2 Hz, 2H), 7.36 (td, J=7.5, 4.4 Hz, 4H), 7.30 (m, 6H), 7.27-7.21 (m, 1H), 6.99 (s, 1H), 5.93 (dd, J=8.3, 4.9 Hz, 1H), 5.03-4.93 (m, 1H), 4.62 (d, J=11.6 Hz, 1H), 4.46 (d, J=11.4 Hz, 1H), 3.95 (d, J=18.3 Hz, 1H), 3.79-3.61 (m, 2H), 3.56 (d, J=14.0 Hz, 1H); ¹³C NMR (126 MHz, DMSO) δ 171.56, 165.03, 160.05, 140.07, 136.26, 129.56, 129.04, 128.90, 128.76, 128.42, 127.34, 127.02, 126.96, 125.18, 122.16, 79.64, 67.17, 58.53, 46.58, 44.23, 41.85; HRMS: Calculated for C₂₉H₂₅CIN₂O₅S ([M+Na]⁺): 571.1065; Found: 571.1060.

### Example 5

Preparation of Compound Benzhydryl 3-(((4-(hydroxymethyl)phenyl)thio)methyl)-8-oxo-7-(2- phenylacetamido)-5-thia-1-azabicyclo[4.2.0]octane-2-carboxylate 5-oxide (6)

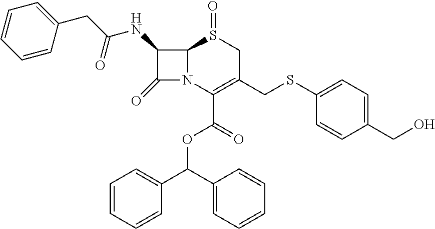

After sonication, a mixture of compound 5 (440 mg, 0.8 mmol) and potassium carbonate (55.2 mg, 1.2 mmol) in anhydrous acetonitrile (10 mL) was stirred at room temperature for 30 min, then (4-mercaptophenyl)methanol (110.4 mg, 0.8 mmol) was added and the resulting mixture was stirred at room temperature for overnight. EtOAc (100 mL) and H₂O (20 mL) was added to the reaction solution, and the separated organic layer was further washed with H₂O (20 mL) twice and saturated brine (20 mL). After removal of the solvent under Rota-Vap, the crude product was purified by flash chromatography with a silica gel column (v/v 0-8% MeOH in DCM) to afford compound 6 (235 mg, 45%).

¹H NMR (500 MHz, DMSO) δ 8.42 (d, J=8.4 Hz, 1H), 7.50 (d, J=7.4 Hz, 2H), 7.36 (s, 3H), 7.32 (d, J=8.4 Hz, 4H), 7.31-7.26 (m, 8H), 7.17 (d, J=2.0 Hz, 4H), 6.80 (s, 1H), 5.85 (dd, J=8.3, 4.8 Hz, 1H), 4.90 (d, J=3.5 Hz, 1H), 4.35 (s, 2H), 4.30 (d, J=13.4 Hz, 1H), 3.95 (d, J=18.4 Hz, 1H), 3.80 (d, J=18.8 Hz, 1H), 3.76 (d, J=13.4 Hz, 1H), 3.68 (d, J=14.0 Hz, 1H), 3.56 (d, J=14.0 Hz, 1H); ¹³C NMR (126 MHz, DMSO) δ 171.48, 164.77, 160.26, 142.26, 140.22, 140.13, 136.16, 132.11, 132.00, 131.39, 129.57, 128.99, 128.84, 128.75, 128.30, 127.55, 127.35, 127.01, 126.90, 124.26, 123.77, 79.30, 62.69, 58.28, 47.46, 41.92; HRMS: Calculated for C₃₆H₃₅N₂O₆S₂ ([M+H]⁺): 655.1920; Found: 655.1931.

### Example 6

Preparation of Compound 10-(((4-(((2-((benzhydryloxy)carbonyl)-5-oxido-8-oxo-7-(2- phenylacetamido)-5-thia-1-azabicyclo[4.2.0]oct-2-en-3-yl)methyl)thio)benzyl) oxy)carbonyl)- 10H-phenoxazine-3,7-diyl diacetate (7)

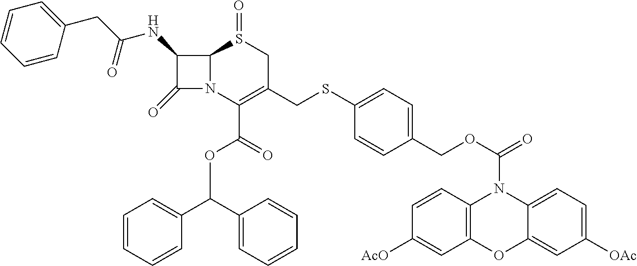

Under an argon atmosphere, to a solution of K₂CO₃ (31 mg, 0.23 mmol) and N,N-dimethyl-4-aminopyridine (3 mg, 0.04 mmol) in DCE (5 mL) was in dropwise added a solution of compound 6 (105.9 mg, 0.162 mmol) in DCE (5 mL) at 0° C. After 30 min, compound 3 (62 mg, 0.17 mmol) dissolved in DCE (10 mL) was added dropwise and the resulting mixture was stirred at room temperature for another 5 hours, monitored by TLC. Then the mixture was diluted with DCM (50 mL) and extracted with water (60 mL) and brine (60 mL) three times. The organic layer was dried with Na₂SO₄, concentrated, and flash silica-gel column (5% methanol in DCM) gave crude compound 7 which was used in the next step without further purification.

### Example 7

Preparation of Compound 10-(((4-(((2-((benzhydryloxy)carbonyl)-8-oxo-7-(2- phenylacetamido)-5-thia-1-azabicyclo[4.2.0]oct-2-en-3-yl)methyl)thio) benzyl)oxy)carbonyl)- 10H-phenoxazine-3,7-diyl diacetate (8)

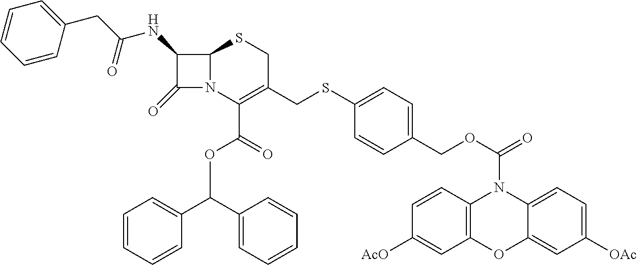 [SMILES: CC(=O)Oc1ccc2c(c1)Oc1cc(OC(C)=O)ccc1N2C(=O)OCc1ccc(SCC2=C(C(=O)OC(c3ccccc3)c3ccccc3)N3C(=O)[C@@H](NC(=O)Cc4ccccc4)[C@H]3SC2)cc1]

The reduction of compound 7 was performed according to a method previously reported. Under argon protection, to a mixture of 7 and Nal (30.1 mg, 0.2 mmol) in anhydrous acetone (10 mL) at ice bath was added dropwise trifluoroacetic anhydride (TFAA) (34 μL, 0.25 mmol). The resulting mixture was stirred at 0° C. for one hour. After removal of the solvent under Rota-Vap, the residue was dissolved in NaHCO₃ (aq.) (5 mL) and extracted with ethyl acetate (5 mL×3). The titled product was purified by a silica gel column (v/v 0-8% MeOH in DCM) as solid (38 mg) at an overall yield of 25% from compound 6.

¹H NMR (500 MHz, DMSO) δ 9.17 (s, 1H), 7.59 (d, J=8.9 Hz, 2H), 7.33-7.25 (m, 19H), 7.04 (d, J=2.6 Hz, 2H), 6.95 (d, J=2.5 Hz, 2H), 6.85 (s, 1H), 6.57 (s, 1H), 5.47 (s, 1H), 5.34 (s, 1H), 5.24 (s, 2H), 5.12 (d, J=3.9 Hz, 1H), 3.94 (q, 1H), 3.63 (s, 1H), 3.51 (d, J=4.5 Hz, 2H), 2.25 (s, 6H); ¹³C NMR (126 MHz, CDCl₃) δ 171.53, 169.13, 166.25, 164.17, 153.01, 150.41, 148.61, 138.85, 134.69, 134.64, 133.62, 132.32, 131.83, 131.49, 129.86, 129.42, 129.17, 129.02, 128.91, 128.68, 128.47, 128.13, 127.57, 127.19, 126.78, 126.40, 125.81, 125.78, 125.74, 125.42, 125.10, 119.63, 118.83, 117.06, 116.31, 110.98, 110.21, 79.57, 68.41, 67.96, 67.29, 60.43, 60.02, 53.93, 53.12, 52.67, 50.72, 50.33, 49.64, 43.04, 40.34, 21.14, 21.00; HRMS: Calculated for C₅₃H₄₃N₃NaO₁₁S₂ ([M+Na]⁺): 984.2231; Found: 984.2221.

### Example 8

Preparation of Compound 3-(((4-(((3,7-diacetoxy-10H-phenoxazine-10- carbonyl)oxy)methyl)phenyl)thio)methyl)-8-oxo-7-(2-phenylacetamido)-5-thia-1- azabicyclo[4.2.0]oct-2-ene-2-carboxylic acid (CDA)

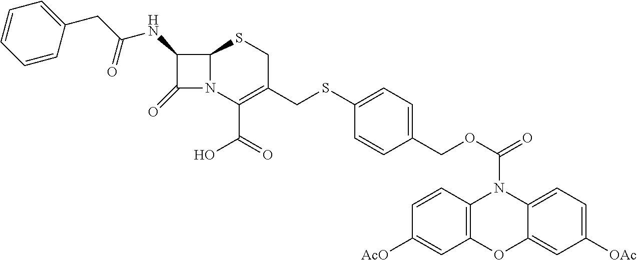 [SMILES: CC(=O)Oc1ccc2c(c1)Oc1cc(OC(C)=O)ccc1N2C(=O)OCc1ccc(SCC2=C(C(=O)O)N3C(=O)[C@@H](NC(=O)Cc4ccccc4)[C@H]3SC2)cc1]

A pre-cold mixed solution of DCM (10.5 mL), trifluoroacetic acid (TFA, 3 mL) and triisopropylsilane (TIPS, 1.5 mL) was added to a flask pre-filled with compound 8 (25 mg, 0.026 mmol). The resulting mixture was then stirred at room temperature for 1 hour. After condensation under reduced vacuum, the crude was purified by HPLC (0.1% acetonitrile from 40% to 100%) to afford probe CDA (10 mg, 48%).

¹H NMR (600 MHz, DMSO) δ 9.14 (d, J=7.7 Hz, 1H), 7.60 (d, J=8.9 Hz, 2H), 7.39-7.34 (m, 4H), 7.28 (t, J=7.4 Hz, 2H), 7.24 (d, J=6.6 Hz, 2H), 7.21 (d, J=7.2 Hz, 1H), 7.04 (d, J=2.6 Hz, 2H), 6.96 (dd, J=8.8, 2.6 Hz, 2H), 6.50 (s, 2H), 5.41 (dd, J=7.7, 3.9 Hz, 1H), 5.24 (s, 2H), 5.13 (d, J=3.8 Hz, 1H), 5.07 (d, J=1.7 Hz, 1H), 4.00 (d, J=14.3 Hz, 1H), 3.77 (d, J=14.2 Hz, 1H), 3.50 (d, J=4.8 Hz, 2H), 2.25 (s, 6H) ; ¹³C NMR (126 MHz, CDCl₃) δ 171.56, 169.72, 168.58, 164.20, 153.04, 150.40, 148.52, 134.95, 134.36, 133.58, 131.42, 129.49, 129.10, 128.31, 127.69, 125.76, 125.35, 119.67, 118.48, 116.96, 110.50, 68.01, 60.16, 53.14, 49.87, 43.10, 40.05, 29.97, 29.72, 21.14; HRMS: Calculated for C₄₀H₃₄N₃O₁₁S₂ ([M+H]⁺): 796.1629; Found: 796.1619.

### Example 9

Expression, purification and SDS-PAGE analysis of β-lactamases: A single colony of E. coli (BL21 or TOP10) containing pBAD-TEV(Cys)-TEM1, pBAD-IMP1-6xHis, pBAD-AmpC-6xHis, pBAD-KPC3-6xHis and pBAD-OXA48-6xHis was inoculated into 100 ml of Lysogeny broth (LB broth) with 100 μg/mL ampicillin, followed by incubation at 37° C., 205 rpm overnight. The overnight culture was added into 500 mL of fresh LB broth with antibiotic and 0.2% arabinose. After incubation for up to 6 h at 30° C., 205 rpm, bacteria were harvested, washed, and the pellet was frozen at −80° C. The pellets were later resuspended and lysed in Novagen Bugbuster protein extraction reagent (EMD Millipore, Burlington, MA). Proteinase inhibitor cocktail (complete, mini, EDTA-free, Roche) was added to the bacterial lysate before two rounds of metal-affinity purification using TALON metal affinity resin. The affinity purified fraction was eluted using lysis buffer containing up to 500 mM imidazole. Buffer was changed to PBS (supplemented with 10% glycerol) by centrifugation with centrifugal filter units (EMD Millipore, Burlington, MA) at regenerated size 30K. Protein samples were denatured in LDS loading buffer (Life Technologies, Carlsbad, CA) with a heating block and analyzed by SDS-PAGE (NuPAGE, Life Technologies, Carlsbad, CA). The gels were stained by SimpleBlue SafeStain protein gel staining reagent. BlaC was expressed and purified as previously described.⁵

### Example 10

Bacteria growth and assay: E. coli (BL21 or TOP10) transformed to express TEM-1 Bla, IMP-1, and KPC3 were grown in LB medium and induced with 0.2% arabinose for 6-8 h at 30° C., 205 rpm. Colony forming units per milliliter (c.f.u./mL) were determined by measuring the UV absorbance at OD₆₀₀.

For CDA incubation, 10 μM working solution was prepared freshly by diluting stock solution (1 mM in pure DMSO) in PBS (pH 7.05). A light-safe tube or foil wrap was used to prevent photobleaching.

Clinically isolated K. pneumoniae, K. pneumoniae expressing KPC, E. cloacae expressing IMP, E. coli expressing OXA-48 and S. marcescens expressing SME were cultured in BD Columbia agar plate containing 5% sheep blood. Resistant bacteria were further inoculated in nutrient broth containing 100 μg/mL imipenem to prevent the loss of resistance.

### Example 11

Statistical Analysis: GraphPad Prism 7 was used for plotting and statistical analysis. The statistical difference was determined by performing two-way ANOVA (FIG. 3d) followed by Bonferroni's multiple comparison test to determine the statistical significance with 95% confidence intervals with *p<0.0332; **p<0.0021, ***p<0.0002, ****p<0.0001, ns: not significant.

1. A dual-caged cleavable probe comprising a b-lactamase- or carbapenemase-cleavable moiety conjugated to a self-immolative linker, wherein the self-immolative linker is conjugated to a fluorescently detectable label.

2. The probe of claim 1, wherein the probe has the formula:
R₁-b-lactam-self-immolative linker-fluorescently detectable label, wherein R₁ is H, a halogen, a substituent selected from NO₂, CN, carboxyl, OH, amine, a substituted or unsubstituted alkyl, a substituted or unsubstituted aryl, a substituted or unsubstituted cycloalkyl, a substituted or unsubstituted cycloalkenyl, a substituted or unsubstituted aryl, a substituted or unsubstituted biaryl, a substituted or unsubstituted fused aryl, a substituted or unsubstituted alkenyl, or a substituted or unsubstituted alkynyl;
the b-lactam is a cephalosporin or a carbapenem, wherein the cephalosporin comprises the structure (a) or (b) and the carbapenem has the structure (c) 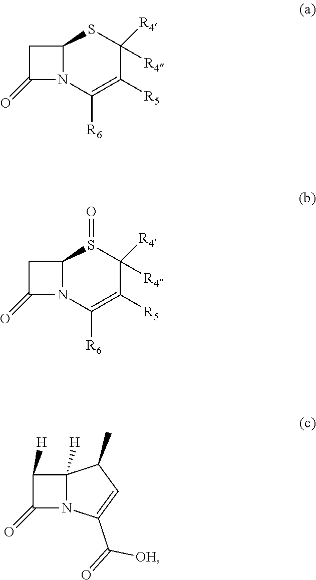
wherein R_{4′}, R_{4″}, R₅, and R₆ are each independently a H, a halogen, a substituent selected from NO₂, CN, carboxyl, OH, an amine, a substituted or unsubstituted alkyl, a substituted or unsubstituted aryl, a substituted or unsubstituted cycloalkyl, a substituted or unsubstituted cycloalkenyl, a substituted or unsubstituted aryl, a substituted or unsubstituted biaryl, a substituted or unsubstituted fused aryl, a substituted or unsubstituted alkenyl, or a substituted or unsubstituted alkynyl; the self-immolative linker has the structure: 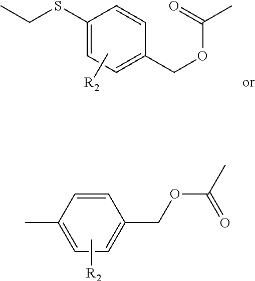
wherein R₂, is a H, a halogen, a substituent selected from NO₂, CN, carboxyl, OH, an amine, a substituted or unsubstituted alkyl, a substituted or unsubstituted aryl, a substituted or unsubstituted cycloalkyl, a substituted or unsubstituted cycloalkenyl, a substituted or unsubstituted aryl, a substituted or unsubstituted biaryl, a substituted or unsubstituted fused aryl, a substituted or unsubstituted alkenyl, or a substituted or unsubstituted alkynyl.

3. The probe of claim 1, wherein the fluorescently detectable label has the structure: 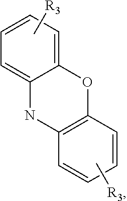
wherein R₃ is a hydrogen, a halogen, a substituent selected from NO₂, CN, carboxyl, OH, an amine, a substituted or unsubstituted alkyl, a substituted or unsubstituted aryl, a substituted or unsubstituted cycloalkyl, a substituted or unsubstituted cycloalkenyl, a substituted or unsubstituted aryl, a substituted or unsubstituted biaryl, a substituted or unsubstituted fused aryl, a substituted or unsubstituted alkenyl, or a substituted or unsubstituted alkynyl.

4. The probe of claim 2, wherein the carbapenem is selected from the group consisting of biapenem, ertapenem, doripenem, imipenem, and panipenem:

5. The probe of claim 1, wherein the probe is a dual-caged N-cephalosporin-caged 3,7-diesterphenoxazine beta-lactamase-cleavable probe or a dual-caged carbapenem-caged 3,7-diesterphenoxazine carbapenemase-cleavable probe.

6. The probe of claim 2, wherein the dual-caged N-cephalosporin-caged 3,7-diesterphenoxazine beta-lactamase-cleavable probe has the structure of formula I:

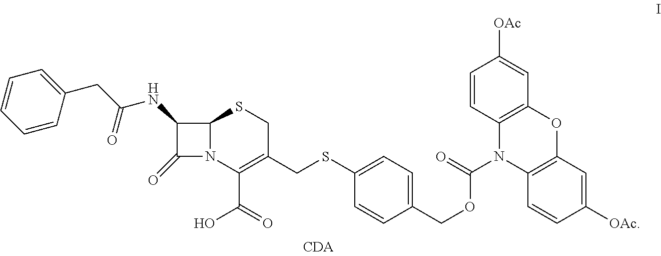 [SMILES: CC(=O)Oc1ccc2c(c1)Oc1cc(OC(C)=O)ccc1N2C(=O)OCc1ccc(SCC2=C(C(=O)O)N3C(=O)[C@@H](NC(=O)Cc4ccccc4)[C@H]3SC2)cc1]

7. The probe of claim 2, wherein the dual-caged carbapenem-caged 3,7-diesterphenoxazine carbapenemase-cleavable probe has the structure of formula II:

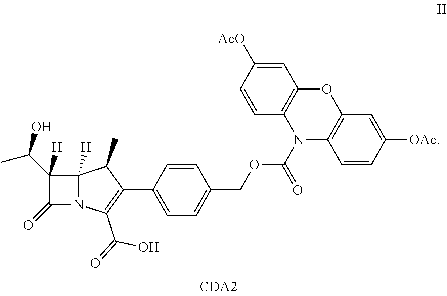 [SMILES: CC(=O)Oc1ccc2c(c1)Oc1cc(OC(C)=O)ccc1N2C(=O)OCc1ccc(C2=C(C(=O)O)N3C(=O)[C@H]([C@@H](C)O)[C@H]3[C@H]2C)cc1]

8. The composition of claim 1, wherein the detectable label is 10H-phenoxazine-3,7-diyl diacetate (2-DARR).

9. A method of detecting the presence of a bacterial strain that has resistance to at least one of a beta-lactam antibiotic or a carbapenem antibiotic, wherein said method comprises contacting a population of bacteria suspected of being resistant to at least one of a beta-lactam antibiotic or a carbapenem antibiotic with a dual-caged N-cephalosporin-caged 3,7-diesterphenoxazine beta-lactamase-cleavable probe or a dual-caged carbapenem-caged 3,7-diesterphenoxazine carbapenem-cleavable probe in the presence of an oxidizing agent, and measuring a fluorescent signal, wherein a detectable fluorescent signal indicates that the bacterial strain has at least one of a beta-lactamase or a carbapenemase.

10. The method of claim 9, wherein the dual-caged N-cephalosporin-caged 3,7-diesterphenoxazine beta-lactamase-cleavable probe or a dual-caged carbapenem-caged 3,7-diesterphenoxazine carbapenem-cleavable probe comprises a b-lactamase- or carbapenemase-cleavable moiety conjugated to a self-immolative linker, wherein the self-immolative linker is conjugated to a fluorescently-detectable label moiety.

11. The method of claim 9, wherein the fluorescently detectable label moiety is 10H-phenoxazine-3,7-diyl diacetate (2-DARR).

12. The method of claim 9, wherein the dual-caged N-cephalosporin-caged 3,7-diesterphenoxazine beta-lactamase-cleavable probe has the structure of formula I: 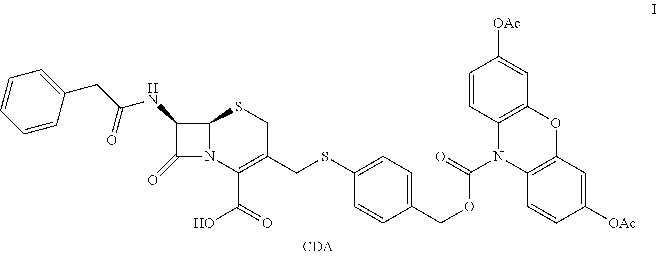 [SMILES: CC(=O)Oc1ccc2c(c1)Oc1cc(OC(C)=O)ccc1N2C(=O)OCc1ccc(SCC2=C(C(=O)O)N3C(=O)[C@@H](NC(=O)Cc4ccccc4)[C@H]3SC2)cc1]
and the dual-caged N-cephalosporin-caged 3,7-diesterphenoxazine beta-lactamase-cleavable probe has the structure of formula II:

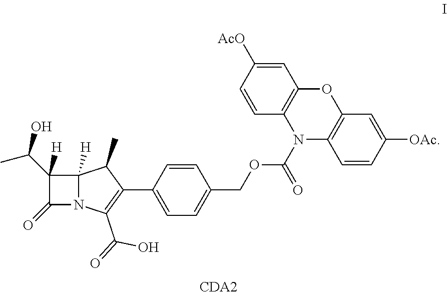 [SMILES: CC(=O)Oc1ccc2c(c1)Oc1cc(OC(C)=O)ccc1N2C(=O)OCc1ccc(C2=C(C(=O)O)N3C(=O)[C@H]([C@@H](C)O)[C@H]3[C@H]2C)cc1]

13. The method of claim 9, wherein the oxidizing agent is hydrogen peroxide.

14. The method of claim 9, further comprising culturing a biological sample from a human or animal to generate a population of bacteria from the sample and concentrating the population of cultured bacteria.

15. (canceled)

16. (canceled)

17. (canceled)

18. (canceled)
